(19) United States
(12) Patent Application Publication
(10) Pub. No.: US 2022/0071547 A1
(43) Pub. Date: Mar. 10, 2022

(54) SYSTEMS AND METHODS FOR MEASURING NEUROTOXICITY IN A SUBJECT

(71) Applicant: Beacon Biosignals, Inc., Boston, MA (US)

(72) Inventors: Jarrett Revels, Brooklyn, NY (US); Matthew S. Alkaitis, Redwood City, CA (US)

(73) Assignee: Beacon Biosignals, Inc., Boston, MA (US)

(21) Appl. No.: 17/469,233

(22) Filed: Sep. 8, 2021

Related U.S. Application Data
(60) Provisional application No. 63/176,589, filed on Apr. 19, 2021.
(60) Provisional application No. 63/075,711, filed on Sep. 8, 2020.

(51) Int. Cl. A61B 5/372; A61B 5/291; A61B 5/00

(52) U.S. Cl. CPC A61B 5/372; G16H 50/20; A61B 5/4094; A61B 5/291

(57) ABSTRACT

The present disclosure relates to a system and method capable of capturing, processing and analyzing electroencephalography (EEG) signals, including features, patterns or signatures with relevance to diagnosis, prognosis, risk stratification or other clinically relevant interpretation, by means of a low-profile head-mounted wireless recording device that may be rapidly applied to the individual being examined. The head-mounted recording device is comprised of an array of electrode elements that contact the subject's forehead, a docking site and an acquisition device that receives, processes, and transmits the EEG data. In one embodiment, the device simultaneously collects additional data, including but not limited to accelerometer data, heart rate, sound level, light level, temperature and/or pulse oximetry. Subsequently, the data is ingested into an analytics system which is capable of identifying features, signatures or patterns that have significance for diagnosis, prognosis, risk-stratification or other medically pertinent observations, estimates or predictions.

### RELATED APPLICATIONS

This Application is a Non-Provisional of Provisional (35 USC 119(e)) of U.S. application Ser. No. 63/176,589, filed Apr. 19, 2021, entitled “LOW-PROFILE FULLY HEAD-MOUNTED WIRELESS EEG RECORDING DEVICE WITH RAPID APPLICATION” and is a Non-Provisional of Provisional (35 USC 119(e)) of U.S. application Ser. No. 63/075,711, filed Sep. 8, 2020, entitled “SYSTEMS AND METHODS FOR MEASURING NEUROTOXICITY IN A SUBJECT”. The entire contents of these applications are incorporated herein by reference in their entirety.

### BACKGROUND

Electroencephalography (EEG) is a method of capturing the electrical activity generated by the nervous system via sensing devices (‘electrodes or other technologies) that are placed on the scalp. Signals are useful clinically as a diagnostic assessment of typical or atypical neural function. One standardized method of electrode placement is to apply scalp electrodes spaced either 10% or 20% of the total width or length of the skull. Placing electrodes in such arrays may require shaving the examinee's head and scraping the skin in order to place electrodes in contact with the skin on the superior or posterior aspects of the head. Various holders have been contrived to arrange and place the electrode, including most commonly for the clinical setting a cap, or a helmet or other structure that configures the location of the set of electrodes.

### SUMMARY

The applicants appreciate that EEG capturing methods and apparatuses are not optimal in their ease of use, among other issues. In particular, it is appreciated that the set-up and acquisition of EEG for clinical studies such as hospital neurological monitoring often requires significant lead-time and can result in discomfort to the examinee. In addition, electrodes that must be tethered to a receiving or signal processing device are often tethered by physical wires, which limit the examinees mobility and can increase risk of delirium or other deleterious effects.

The collection and analysis of EEG signals and other biomarkers associated with EEG data collection (such as the effectiveness of the sensor connection) also requires specialized equipment, skilled technicians, and medical experts to perform data acquisition and interpretation. Further, standard clinical workflows may require medical experts to be on-premise to access and interpret the EEG data. This standard approach often limits the clinical utility of EEG due to extended technical set-up time, significant deleterious impact and discomfort for the patient, ongoing monitoring of the state of the system by trained experts, significant time and expense to arrange for expert interpretation, and delays in returning final interpretations to the primary healthcare provider responsible for the care of the patient to make any relevant clinical decision(s).

Some have attempted to enable more rapid or less burdensome acquisition of EEG data, such as technologies involving dispensing conductive gel from electrode elements (Parvizi et al., 2017). However, such technologies still require operator experience that is often beyond the capabilities of the examinee themselves. Such technologies also typically include physically bulky non-disposable components that may require additional logistical effort to store, manage or return to a vendor prior to re-use for a new exam. As a result, there is a clear need for technologies that are user-friendly to the degree that an untrained individual such as the examinee themselves could apply the device and obtain clinical-grade data. There is also a need for technologies with minimal non-disposable components that would reduce the logistical burden of managing hardware following the completion of the exam. Furthermore, the majority of these technologies tether an individual to a fixed device or have an unwieldy and uncomfortable component attached to the sensors themselves. This makes acquisition of clinical-grade EEG cumbersome for the ambulatory setting. Finally, existing technologies often include custom transmitter hardware components that limit compatibility with commercially available devices such as smartphones, tablets or desktop computers. Such limitations thus require additional hardware to be supplied with the EEG detector device and also increase the burden on the operator or examinee to set up the device and initiate an exam.

Some embodiments described herein include systems and methods including hardware components that enable simple and rapid application of an EEG detection device, which, along with custom software and a computer-readable medium, is capable of capturing, processing, storing and analyzing electroencephalography (EEG) signals, including features, patterns or signatures with relevance to diagnosis, prognosis, risk stratification or other clinically relevant interpretation. In some embodiments, a head-mounted recording device is provided that includes a low-profile array of electrode elements that adhere to the subject's forehead. In some embodiments, the device includes a docking site and an acquisition device that receives, processes, and transmits the EEG data. In some embodiments, the electrode array is disposable, enabling the durable portion of the acquisition device to be returned to the operator or vendor for future use. In other embodiments, the entire EEG detection device is disposable. The EEG detection device is also capable of simultaneously collecting additional data, including but not limited to accelerometer data, heart rate, temperature and/or pulse oximetry, among other data.

The data is ingested into an analytics system which is capable of identifying features, signatures or patterns that have significance for diagnosis, prognosis, risk-stratification or other medically pertinent observations, estimates or predictions. It should be appreciated that portions of the analytics may be performed on the head-mounted device, a separate computer system, or combinations thereof. This analysis may be performed with or without the use of electronic medical record data, ingestion of other data streams (e.g., heart rate, temperature, or pulse oximetry) and/or a corpus of previously collected EEG data. A display device enables the operator to provide input to set the parameters of the exam. The same or a different display device provides real-time data feeds or summary analysis to an expert for interpretation and/or a healthcare provider responsible for care of the subject and/or the subject themselves for interpretation. The system is also capable of generating comprehensive electronic reports describing the results of relevant analyses that have been performed with or without confirmation by a licensed practitioner.

In some embodiments, the device is configured to record and analyze EEG signals from the brains of patients in multiple clinical settings including but not limited to those with possible seizures, altered mental status, sleep monitoring, intoxication, being treated with anesthetic agents and in other settings in which an EEG would be routinely utilized. The device may also be implemented in clinical settings where EEG may be used to study, validate, or implement novel clinical neurobiomarkers, defined as signatures that have been identified to correlate, predict or otherwise associate with clinical observations, disease processes or outcomes of interest. Based on these capabilities, the device may be used to perform diagnosis of disease states, monitoring of therapeutic effect from interventions, monitoring of side effects or real-time or intermittent assessment of clinical trajectory or prognosis in patient populations ranging from in-hospital patients to at-home studies or characterization of normal brain physiology.

In one exemplary embodiment of the disclosure, the system and method described above may be used to provide EEG data directly to a qualified expert for interpretation. In such an embodiment, the disclosed method may additionally generate visual or digital guide elements to assist the qualified expert in navigating the data to identify relevant features, signatures, or other findings.

In another exemplary embodiment of the disclosure, the system and method described above may perform relevant analyses without direct input from a qualified expert. In this embodiment, machine learning algorithms may be trained on a corpus of existing EEG data to identify relevant features, signatures or other findings. These analyses may be performed with or without additional data obtained via integration with the subject's electronic medical record, via data input by the subject with the display device, via data input by the technician or operator with the display device or via ingestion of additional data streams such as heart rate, pulse oximetry, temperature, gyroscopic or accelerometer data that may be collected by the head-mounted device or separate sensors.

According to one aspect, an electroencephalography (EEG) detection system is provided. The system comprises of a wearable head-mounted device, comprising of a plurality of sensors arranged at different locations, with each sensor configured to capture electrical signals from a portion of a body of an examinee, a data acquisition apparatus configured to process electrical signals from the sensors and wirelessly transmit said electrical signals to a receiver device, the receiver device being configured with one or more processors to receive and process data transmitted by the acquisition device, and one or more computer storage media to store data generated by the head-mounted device.

According to one embodiment, the system further comprises of a display device configured to present virtual content to the examinee, examiner or other operator. According to one embodiment, said data acquisition apparatus continuously transmits said EEG signals to said receiver device. According to one embodiment, said data acquisition apparatus is capable of being configured wirelessly. According to one embodiment, a flexible material is used to connect the sensors in a pre-specified arrangement such that the examinee, examiner or other operator may rapidly apply the sensors at the desired locations without having to place each sensor individually. According to one embodiment, said flexible material and said sensors may be manufactured in a plurality of arrangements, including variation in number and/or position of sensors, that may be interchangeably attached to the data acquisition device according to examinee characteristics, desired exam or clinical use case. According to one embodiment, said head-mounted electrode array and said acquisition device are integrated into the same device. According to one embodiment, said display device and said receiver device configured with one or more processors are the same device. According to one embodiment, the head-mounted device is further configured with one or more sensors from the group comprises of an oximeter, a temperature sensor, a gyroscope, an accelerometer, and a heart rate monitor. According to one embodiment, said one or more computer storage media are further configured to store one or more from the group comprises of a database for quality comparison, a database for feature identification, a database for pattern identification, and a database for patient stratification and population analysis. According to one embodiment, said one or more processors are further configured to perform operations comprises of accessing one or more from the group comprising of a database for quality comparison, a database for feature identification, a database for pattern identification, and a database for patient stratification and population analysis, assessing the quality of exam data, extracting or identifying features from the exam data, extracting or identifying patterns from the exam data, and extracting or identifying patient stratification or other population analysis.

According to one embodiment, responsive to the collection of additional data from the one or more examination devices or sensors, the one or more processors are further configured to perform operations comprising of one or more from the group comprises of creating a report summarizing the results of exam data analysis, creating data visual guide elements that are displayed to the user, creating data annotations that are stored along with the underlying data in a non-transitory computer-readable medium, and configuring the head-mounted device to provide feedback to one or more users in the form of light, sound, or vibration. According to one embodiment, the one or more processors are further configured to perform train a machine learning or statistical model on data stored in a non-transitory computer-readable medium, and the one or more computer media are further configured to store the model architecture, parameter values and any other variables required to implement said machine learning or statistical model. According to one embodiment, responsive to the collection of additional data from the one or more examination devices or sensors, the one or more processors are further configured to re-train said machine learning or statistical model with an updated data set.

According to one aspect, a non-transitory computer-readable medium storing one or more instructions that, when executed by one or more processors, cause the one or more processors to perform operations is provided. The non-transitory computer-readable medium comprises of accessing one or more from the group comprising of a database for quality comparison, a database for feature identification, a database for pattern identification, and a database for patient stratification and population analysis, assessing the quality of exam data, extracting or identifying features from the exam data, extracting or identifying patterns from the exam data, and extracting or identifying patient stratification or other population analysis.

According to one embodiment, responsive to the collection of additional data from the one or more examination devices or sensors, said non-transitory computer-readable medium stores further instructions that, when executed by one or more processors, cause said one or more processors to perform operations comprising of one or more from the group comprises of creating a report summarizing the results of exam data analysis, creating data visual guide elements that are displayed to the user, creating data annotations that are stored along with the underlying data in a non-transitory computer-readable medium, and configuring the head-mounted device to provide feedback to one or more users in the form of light, sound, or vibration. According to one embodiment, when executed by one or more processors, cause said one or more processors to train a machine learning or statistical model on data stored in a non-transitory computer-readable medium and store the model architecture, parameter values and any other variables required to implement said machine learning or statistical model. According to one embodiment, responsive to the collection of additional data from the one or more sensors, said non-transitory computer-readable medium further stores instructions that, when executed by one or more processors, cause said one or more processors to re-train said machine learning or statistical model with an updated data set. According to one embodiment, the non-transitory computer-readable medium further comprises of a remote data repository comprising the one or more computer storage media storing the computer readable instructions and a remote processing module comprising the one or more processors, wherein the one or more processors are configured to perform the operations.

According to one aspect, a method is provided. The method comprises of determining the type of medical examination desired, initiating collection and storage of data streams from said system, collecting and/or storing subsequent data streams from said system, accessing one or more from the group comprising of a database for quality comparison, a database for feature identification, a database for pattern identification; and a database for patient stratification and population analysis, assessing the quality of exam data, extracting or identifying features from the exam data, extracting or identifying patterns from the exam data, creating a report summarizing the results of exam data analysis, creating data visual guide elements that are displayed to the user, creating data annotations that are stored along with the underlying data in a non-transitory computer-readable medium, and configuring the head-mounted device to provide feedback to one or more users in the form of light, sound, or vibration.

According to one aspect, an electroencephalography (EEG) detection system is provided. The system comprises of a wearable head-mounted device, comprising of a processor, a memory coupled to the processor, and an array of sensors, operatively coupled to the processor and configured to capture electrical signals from a forehead area of a subject, wherein the array of sensors are located at fixed positions and are capable of being attached to the forehead area.

According to one embodiment, array of sensors are positioned within a flexible component that are capable of being attached to the forehead area by an adhesive layer. According to one embodiment, the sensors in the array of sensors includes an electrically conductive component configured to detect electrochemical depolarizations of underlying central nervous system tissue of the subject. According to one embodiment, the array of sensors includes at least a first electrode positioned within the flexible component in a location that aligns substantially with a midline of the subject's forehead area. According to one embodiment, the array of sensors includes at least a second and third electrode, each of which is positioned within the flexible component at lateral left and right locations relative to the midline of the subject's forehead area. According to one embodiment, the array of sensors is a disposable item. According to one embodiment, the array of sensors is detachable from the processor and memory.

According to one embodiment, the wearable head-mounted device further comprises a visual indicator that is configured to provide a visual indication to a person monitoring the subject. According to one embodiment, the wearable head-mounted device further comprises an audio sensor that is configured to detect ambient sound in an environment of the subject. According to one embodiment, the wearable head-mounted device further comprises a light sensor that is configured to detect ambient light in an environment of the subject. According to one embodiment, the array of sensors includes a particular arrangement of sensors for a corresponding particular clinical use. According to one embodiment, the array of sensors is configured to be applied to desired locations on the subject's forehead without having to place each sensor individually. According to one embodiment, the wearable head-mounted device is configured to provide feedback to the subject in the form of light, sound, and/or vibration. According to one embodiment, the electrical signals are EEG signals. According to one embodiment, the head-mounted device is further configured with one or more sensors from the group comprising of an oximeter, a temperature sensor, a gyroscope, an accelerometer, and a heart rate monitor.

According to one aspect, an electroencephalography (EEG) detection system is provided. The system comprises of a wearable head-mounted device, comprising of a plurality of sensors arranged at different locations on a subject, with each sensor configured to capture EEG signals from the subject, a data acquisition device configured to process electrical signals from the sensors and transmit said EEG signals to a receiver configured with one or more processors to receive and process data transmitted by the acquisition device, and a machine learning engine configured to receive and process the EEG signals and perform at least one of the group of operations comprising of an automatically identify patterns within the received EEG signals, automatically annotate at least a portion of an EEG waveform, control a visual indicator to signal an examiner of the subject of a particular condition of the subject, indicate a quality of the EEG signals, and control a feedback generator to provide feedback to the subject in the form of at least one of the group comprising light, sound, and vibration.

According to one embodiment, the machine learning engine is configured to receive and process signals from one or more of the sensors from a group comprising of an oximeter, a temperature sensor, a gyroscope, an accelerometer, and a heart rate monitor.

This summary is a simplified and condensed description of the underlying conceptual framework, enabling technology, and possible embodiments, which are further explained in the detailed description below. This summary is not intended to provide a comprehensive description of essential features of the invention, nor is it intended to define the scope of the claimed subject matter.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF DRAWINGS

Various non-limiting embodiments of the technology will be described with reference to the following figures. It should be appreciated that the figures are not necessarily drawn to scale. In the figures:

FIG. 1 is a diagram depicting one exemplary set of components capable of implementing the system and method. In this example, the display device, remote processing module, electronic medical record, database(s) or data repositories, other detector system(s) and computing or analytics system(s) interact by means of a communication network or networks.

FIGS. 2A-B show a block diagram depicting an exemplary set of functional components, including embodiments of processing environment components capable of implementing various embodiments.

FIG. 3A is a diagram showing the rapid application of the electrode array to the patient or subject's forehead and docking of the acquisition device via an example acquisition device docking apparatus.

FIG. 3B is a head-on view of an exemplary set of hardware components comprising the forehead-mounted electrodes and docking apparatus.

FIG. 3C is a detailed view of an exemplary construction of printed electrode elements.

FIG. 3D is a detailed view of an exemplary assembly of non-printed electrode elements.

FIGS. 3E-F show an exploded (3E) and an assembled view (3F) of the docking apparatus that seats an example acquisition device.

FIGS. 4A-C show a side-angle assembled view (4A), an exploded view (4B) and an inferior view (4C) of a charging apparatus according to one embodiment.

FIG. 4D is a view of the charging apparatus circuit board

FIGS. 4E-F show an exploded view (4E) and an assembled view (4F) of a data acquisition device according to some embodiments.

FIG. 5A is a diagram showing one embodiment of a local processing unit within an acquisition device.

FIG. 5B is a block diagram that depicts an embodiment of the software architecture that enables the amplifier and transmitter device.

FIG. 6 is a flow diagram showing the acquisition and pre-processing of EEG data by the local processing unit within the acquisition device.

FIG. 7 is a block diagram that depicts an embodiment of the software architecture that enables the receiver device.

FIG. 8A is a diagram demonstrating multiple methods of enabling pairing between the display and receiver device and the EEG acquisition device.

FIG. 8B is a diagram demonstrating feedback display elements that enable the examiner, examinee or other operator to interpret the current state of the acquisition device.

FIGS. 9A-B show a flow diagram demonstrating the initiation of an exam and collection of exam data enabled by the disclosed system and method.

FIG. 10 is an exemplary embodiment of the display interface that would enable an expert to interrogate the data captured by the detection device with the assistance of virtual guide elements and the ability to annotate or confirm diagnostic interpretations.

FIGS. 11A-I show examples of virtual guide elements that are used to identify features or signatures for the purpose of identifying diagnostically relevant signals or patterns.

FIG. 12 shows an example of a process for detecting neurotoxicity in a patient/subject of a clinical trial using EEG data.

### DETAILED DESCRIPTION

Some embodiments discussed herein comprises a system and method of obtaining, processing and analyzing an EEG signal in order to do one, two, or all of: simplifying the application of an EEG acquisition device, reducing examinee discomfort, improving the quality or quantity of the data acquired during the examination, reducing inter-operator variability, decreasing interpretation turn-around time, and/or identifying features, signatures, patterns or characterizations that were previously difficult or impossible to reproducibly assess by qualified experts.

In some embodiments, a system is provided that includes a head-mounted EEG detector device, including an array of electrodes that adhere to the forehead, as well as zero, one, or a plurality of medical examination devices that include but are not limited to, pulse oximeters, temperature probes, accelerometers, gyroscopes etc. One or more components of the head-mounted device may be disposable. Such a system may also include a display device to gather input from the subject or the operator about zero, one, or more of the following: the exam being performed, specific symptoms the subject is experiencing, biometric data or other relevant medical or personal data. During an examination, the head-mounted EEG detection device ingests, processes and transmits EEG data and/or data from other sensors. The method by which local processing is performed may or may not be updated throughout the examination based on feedback from the local processor or via a communications network such as Bluetooth or Wi-Fi.

At least some methods utilized herein includes analysis of raw or processed EEG data to generate virtual guiding elements to guide review, annotation or interpretation of the EEG data by a non-expert or a qualified expert such as a medical doctor. These virtual guiding elements may include but are not limited to instruction in the form of plain text or otherwise, one or more arrows, targets, circles, color-changing elements, progress bars, transparent elements, angles, projections of real-time or stored EEG or sensor data, or other virtual items. These virtual items may be related to features or signatures that may be previously known to the field via publication, training, word-of-mouth or expert annotations. Alternatively, these virtual items may be related to novel features, signatures or elements that are extracted algorithmically from individual EEG exam data or from a corpus of previous or simultaneous EEG exams.

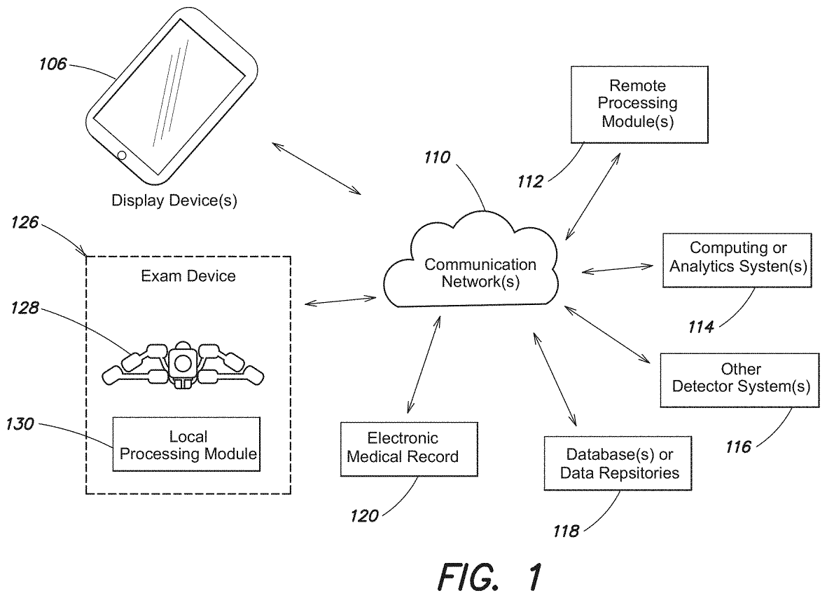

FIG. 1 depicts an exemplary set of hardware and functional components capable of implementing at least some embodiments described herein. In this example, the head-mounted EEG detector device 126 includes a local processing module 130 that interacts with a communications network 110 or plurality of networks such as Bluetooth or Wi-Fi to communicate with the display device 106, remote processing module 112, electronic medical record 120, database(s) or data repositories 118, other detector system(s) 116 and computing or analytics system(s) 114.

To use this hardware system, the examinee or an examiner, who might be a health care provider, technician or other operator would apply the EEG detection device electrode array 128 to the examinee's forehead. In one embodiment, the device would initiate monitoring of the subject or patient's EEG data autonomously. In another embodiment, the operator could use the display device 106 to enter data and/or select and initiate an exam. A computing system to manage data ingestion, processing, management, annotation may include a local processing module 130 which may use Wi-Fi, Bluetooth or other protocol to access a communication network 110 and interface with a remote processing module 112, other computing system(s) 114, other detector system (s) 116, or database(s) or data repositories 118 to perform the acquisition, ingestion and processing of EEG or other sensor data. In one embodiment of the system, the interpretation of the EEG data could be performed by an algorithm stored on a computer-readable medium either locally on the device's local processing module 130 or on a remote processing module 112. In another embodiment, a qualified human expert would review the raw or processed data and would perform the interpretation via the display device 106 or other interface that would enable annotation of features, signatures or other data elements with or without an overall interpretation and summary of the findings. In any of the above embodiments, the final interpretation could be returned to the examinee, the operator or another individual requiring the information via the display device, some other method of electronic communication, or by uploading the report to an electronic medical record 120 associated with the subject.

In one embodiment, the head-mounted EEG detection device 126 includes a plurality of sensors in addition to EEG electrodes including but not limited to: oximetry, sound sensors, light sensors, temperature sensors, gyroscopes, accelerometers and heart-rate sensors. In another embodiment, the device includes a minority of these sensors in addition to the EEG electrodes, or only includes the EEG electrodes with no additional sensors.

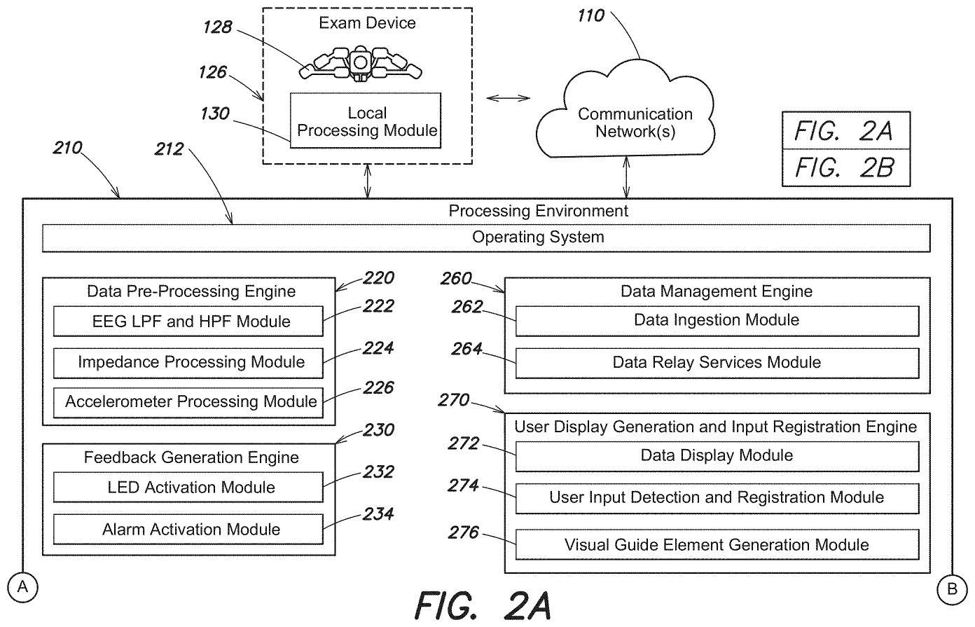

FIGS. 2A-B show a block diagram depicting an exemplary set of functional components, including embodiments of processing environment components capable of implementing various aspects of EEG detection device technology as described herein. In said exemplary set of embodiments, the processing environment 210 may be supported and implemented by either local processing modules 130, or by remote processing module(s) 112 described in FIG. 1 via a communication network 110, or by a combination of local and remote modules. In said embodiments, the processing environment 210 is comprised of an operating system 212, a data pre-processing engine 220, a feedback generation engine 230, a signal processing capture and storage engine 240, a data analysis engine 250, a data management engine 260, a user display generation and input registration engine 270, an application programming interface service 280 and a storage layer 290.

Said operating system 212 establishes the underlying software infrastructure that provides configuration instructions to the hardware components and the local processing module 130 and processes, analyzes, stores and/or otherwise manages data collected by the exam device 126. Components of said processing environment 210 and said operating system 212 may be implemented across multiple processing modules described in FIG. 1. As an exemplary implementation of the disclosed technology, said data pre-processing engine 220 and said feedback generation engine 230 are implemented with the local processing module enabled by the exam device, whereas said user display generation and input registration engine 270 is implemented on the display device 106 described in FIG. 1, and said communications network 110 is used to interface with a remote processing module 112 that implements said signal processing capture and storage engine 240, a remote data repository that implements said storage layer 290, and a remote computing system that implements said data analysis engine 250, said data management engine 260 and said application programming interface service 280. Application programming interface (API) service 280 may include one or more programmatic interfaces through which data may be accessed and stored. Service 280 may include a detector output capture module 282 that functions to capture detector outputs, and service 280 may include an exam progression data capture module that captures information relating to individual examinations.

It should be appreciated that various functions may be performed on or off-device or in combination, and some embodiments are not limited to the location of the function performed. Further, it should be appreciated that certain aspects of the system may include a specially-programmed computer, but in some instances, may be a general purpose computer including standard hardware and operating systems, upon which various component described herein may be implemented.

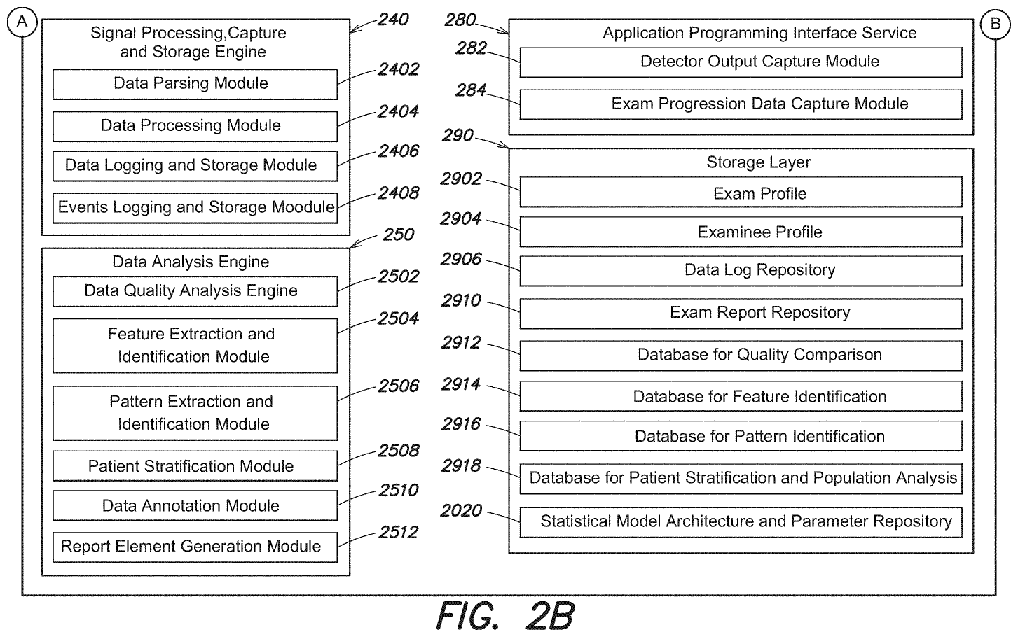

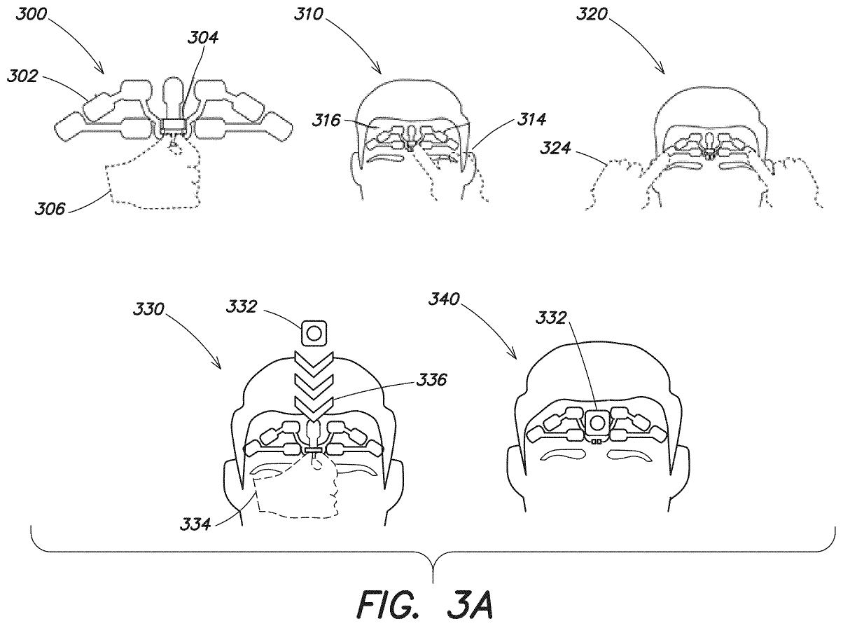

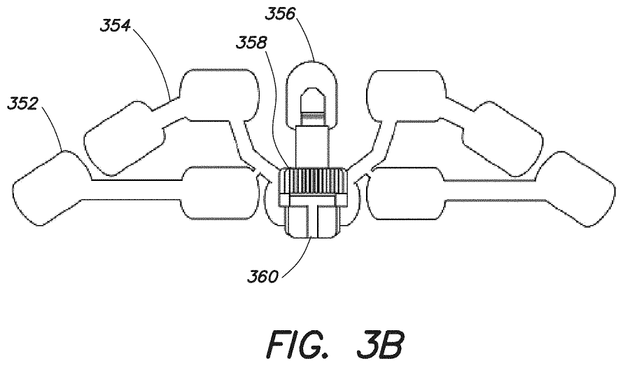

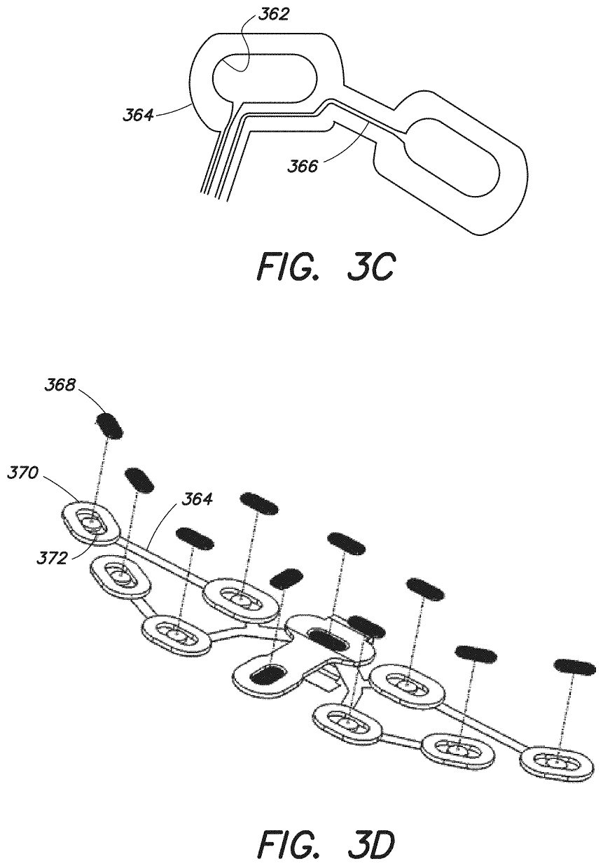

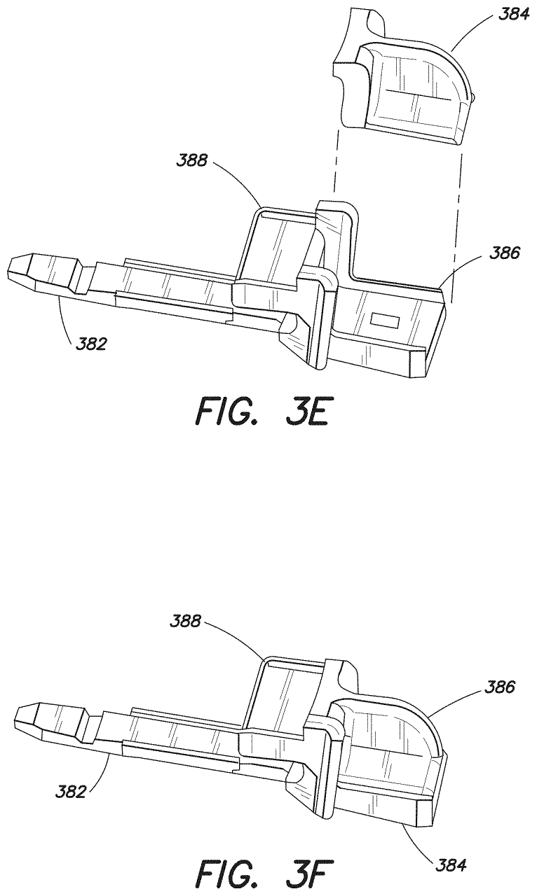

Said data pre-processing engine 220 accesses sensor data that may include but is not limited to EEG, cameras, gyroscopes, accelerometers, oximeters, temperature probes, heart-rate monitors, depth-sensing systems and other sensors or systems. In an exemplary embodiment, an EEG low-pass filter and high-pass filter module pre-processes EEG data collected by the electrode array described in FIG. 3B. In this embodiment, the data pre-processing engine also includes processing modules for impedance 224 and accelerometer 226 data. In other embodiments, additional processing modules are implemented to process data collected from cameras, gyroscopes, oximeters, temperature probes, heart-rate monitors, depth-sensing systems or other sensors or systems.

Said feedback generation engine 230 controls modules that generate feedback signals that are implemented by the hardware components described in FIGS. 3A-5B. In an exemplary embodiment of the disclosed technology, the feedback generation engine 230 enables an LED activation module 232 to control illumination, frequency, duration, color or other parameter for one or a plurality of light-emitting diodes (LEDs) or other light-emitting indicators. In said embodiment, the feedback generation engine 230 also enables an alarm activation module 234 to control other types of feedback signals, including but not limited to sound, buzz, haptic feedback or other signal type that may be interpreted by the examinee, examiner or other operator.

Said signal processing, capture and storage engine 240 enables management of pre-processed data that are generated by the data pre-processing engine 220, or, in other embodiments, data that are collected directly from the EEG electrodes or other relevant sensors. Said signal processing, capture and storage engine 240 enables implementation of a data parsing module 2402 and a data processing module 2404, which prepare data for further analysis or storage. The data logging and storage model 2406 and events logging and storage module 2408 interact with the storage layer 290 to capture and store data and events respectively, as determined by the requirements of the exam, or input from the examiner, examinee or other operator.

Said data analysis engine 250 allows real-time or asynchronous assessment of output from the EEG detector device or other sensor. The data quality analysis module 2502 evaluates the data that is being generated by the EEG detector device or other sensors compared to predetermined thresholds or real-time calculated thresholds that indicate sufficient data quality to enable interpretation or other data processing, storage or analysis tasks. In one embodiment, said evaluation is performed with a real-time updated estimator of data quality and an associated confidence estimator.

The feature extraction and identification module 2504 enables raw, pre-processed or processed data to be evaluated for the presence, frequency, characteristics or other parameters of features. One example of a feature that may be detected is an epileptiform spike. The pattern extraction and identification module 2506 enables raw, pre-processed or processed data to be evaluated for the presence, frequency, or characteristics of patterns that are constructed from multiple features, consist of observable changes over time, or represent a statistical association with a parameter or outcome of interest. One example of a pattern that may be used is burst-suppression, a brain state seen in stages of general anesthesia, coma, or hypothermia. The patient stratification module 2508 enables patient-level data to be evaluated in the context of one or a plurality of other patient-level data in order to identify commonalities, differences, sub-group definitions, sub-group characteristics, or other analyses relevant to features, patterns, statistical associations or other analytical conclusions that may be made about a group of individuals.

The data annotation module 2510 creates data annotations that capture the results of data analysis performed by other elements of the data analysis engine 250, aid the system in future data retrieval and/or aid the examinee, examiner, other operator or qualified expert in reading, surveying, interpreting or otherwise interacting with the data. Said data annotation module may communicate with the visual guide element generation module 276 in order to make said annotations available to the examiner, examinee, other operator or qualified expert. The report element generation module converts the analyses performed by other elements of the data analysis engine 250 into elements that are represented in a report describing the results of the exam, in summary format, with detail about specific analytical results or with a combination thereof. Generated report elements may be created as a printed element, electronically generated in a static or dynamic format, or may be modified into any other format by which the results of the exam may be communicated to the examiner, examinee, other operator, qualified expert, family member or other authorized party.

Said data management engine 260 enables data ingestion and relay from multiple sources. In an exemplary embodiment, the data ingestion 262 and relay services 264 modules enable data and analysis results from the signal processing capture and storage engine 240 and data analysis engine 250 to be relayed to the storage layer 290. In said embodiment, the data ingestion module 262 also enables ingestion of EEG or other sensor, laboratory, imaging or other clinical data such that these data may be added to relevant databases in the storage layer 290 and/or accessed by other elements of the processing environment to aid in the performance of analyses, determination of relevant configurations, creation of report elements, or any other action performed by said system.

Said user display generation and input registration engine 270 enables the examinee, examiner, other operator, qualified expert or other authorized party to interact with the system. The data display module 272 generates an interface whereby one or a plurality of said users can view elements including, but not limited to: real-time data streams, results of analyses, warnings or alarms, metrics related to exam quality, metrics related to connectivity, technical information about the system or other information relevant to the performance, management or interpretation of the exam. The user input detection and registration module 274 enables the system to capture a range of inputs from the examinee, examiner, other operator, qualified expert or other authorized party in order to aid the user in making one or a plurality of selections, inputting data, modifying the exam or other functions that require input from the user or aiding the system in the performance of setting exam parameters, setting configuration, data collection, data processing, data analysis, or any other function performed by the system. One or a plurality of said users can provide information via said user input detection and registration module 274 via one or a plurality of interactions including but not limited to: pressing a button, moving a switch, performing a touch-sensitive interaction with a screen, typing information, speaking a command or creating another sound, eye tracking, gesture detection or any other mechanism by which the user communicates information to the system

Said user input detection and registration module 274 also enables one or a plurality of said users to provide information based on prior knowledge, expertise or active problem solving to interpret the data, analysis, report or other artifact generated by the system. In some cases, the information provided by one or a plurality of said users may be to verify the accuracy of the data, analyses or report generated by the system. The visual guide element generation module 276 may utilize data such as information about the exam environment, the examinee, features or signatures within the EEG waveform data or other data available about the patient, exam or data feeds, data collected from the subject's electronic medical record, data stored in the storage layer or other supplementary data. Using these data sources, the visual guide element generation module 276 is able to produce the design, content, appearance and other features of virtual exam guide elements for the purpose of helping the examinee, examiner, other operator, qualified expert or other authorized user to identify relevant features signatures or other observable elements of the data in order to perform an accurate interpretation.

In another embodiment, the interpretation is performed autonomously by an algorithm stored in a computer-readable format or generated de novo for the specified analysis, and the visual elements are used to explain the interpretation that was generated automatically (e.g., through machine learning/AI, or other statistical analysis technique). The visual elements generated by said visual guide element generation module 276 may include but are not limited to text instructions, guiding graphics such as one or more arrows, targets, circles, color-changing elements, progress bars, transparent elements, angles, ‘ghost’ outlines of comparator data signatures, projections of real-time or stored imaging data, or other virtual items. The guiding elements may exist and change according to a predetermined set of instructions, or in response to feedback elements such as navigation through data feeds or new annotations performed by one or a plurality of authorized users, passage of time, acquisition of new exam data, other user input, examinee input. The guiding elements generated by the method may be updated before, during, or after the exam via any of these inputs alone or in combination. The disclosed method may generate or adapt visual or graphical elements in accordance with completion or lack of completion of the exam or a subset thereof.

Storage layer 290 is an integrated data layer that allows high-throughput ingestion, retrieval, and inference of signal data. Encoding is performed by the signal processing and integration engine 240, which in one embodiment of the disclosed method, implements a compression codec that utilizes block-based and pre-entropy-coding inter-channel decorrelation. Said data stored in the storage layer 290 may be used as an input for other functional components of the system. In particular, data may be retrieved from said data analysis engine 250 for inference, interpretation or other subsequent analysis by the signal analysis engine 250.

In one exemplary embodiment, said storage layer 290 maintains a notion of semantically versioned processes that interact with and generate database content (reports, annotations, derived signals/artifacts), a request/response-driven task management workflow for extracting insight from domain experts, a notion of programmable votes/ballots for aggregating expert consensus, and a browser application that facilitates task-oriented machine-learning augmented workflows for biosignal recording labeling, monitoring, and review. Stored data may include, but is not limited to, exam profile data 2902, examinee profile data 2904, a data log repository 2906, an exam report repository 2910, one or a plurality of databases for quality comparison 2912, one or a plurality of databases for feature identification 2914, one or a plurality of databases for pattern identification 2916 and one or a plurality of databases for patient stratification and population analysis 2918, or other stored data types or categories. The statistical model architecture and parameter repository 2920 stores architectures, parameters and any other variables necessary to implement a machine learning or statistical model to identify features, patterns, patient stratification or other clinically relevant read-outs and/or other outputs. Said components of the storage layer may be stored in one or a plurality of distributed machine-readable media connected by a communications network.

FIG. 3A demonstrates the rapid application of the electrode array to the patient or subject's forehead and docking of the acquisition device via the acquisition device docking apparatus. In one exemplary embodiment of the system, the head-mounted EEG detection device includes several components: 1) a set of adhesive flexible electrodes 302 which can be placed on the forehead (EEG “sensors”), 2) a docking apparatus 304 integrated with said electrodes via conductive material and 3) a separate acquisition device 332 that can be docked on the docking apparatus to enable amplification, processing and transmission of the EEG data. The modular design of these components enables the rapid assembly of a functional head-mounted device by the examinee or an operator initiating the exam. The application and assembly of the head-mounted device is intended such that a non-specialized examinee, technician, nurse or doctor could apply the device to the examinee's forehead in one minute or less. Despite this ease-of-use some operators may require additional time to complete application and assembly of the head-mounted device.

Frame 300 demonstrates a first step in applying the head-mounted EEG detection device, whereby the examiner or examinee first grasps the inferior aspect of the integrated docking apparatus 304 using the grasp technique demonstrated in 306 or other approach to holding the device. A protective layer is then removed from the back of the electrode array to reveal adhesive material. As illustrated in frame 310 the electrodes are then ready to apply to the examinee's forehead 316 by approximating the electrodes to the desired position and pressing the middle of the electrode array using the technique demonstrated by 314 such that each electrode is flat and securely adhered to the examinee's skin (e.g., by pressing by hand). As illustrated in frame 320 each lateral electrode is then similarly pressed into place using the technique demonstrated in 324 or similar approach. As illustrated in frame 330, once the full array of electrodes is adequately adhered to the examinee's forehead, the acquisition device 332 can then be docked into the docking device by grasping the docking device using the technique indicated by 334 and pressing downward as indicated by 336. Frame 340 demonstrates the final assembled head-mounted EEG detection device with the acquisition device 332 fully seated into the docking apparatus. In one embodiment of the device, the adequate docking of the acquisition device into the docking apparatus causes the acquisition device to turn on and start collecting EEG signals. The completion of assembly and initiation of data collection may be indicated by a signal including but not limited to: LEDs embedded in the acquisition device, generation of sound or generation of a message, sound or signal on the display device.

In another exemplary embodiment of the system, the acquisition device is fully integrated with the EEG electrode array. In this embodiment, the removal of the protective layer from the back of the electrodes, or the completion of application of each electrode to the examinee's forehead may prompt a signal to be generated by the acquisition device, initiation of an exam or other action by the system.

FIG. 3B is a head-on view of an exemplary set of hardware components comprising the lightweight forehead-mounted electrodes and docking apparatus. This component of the device is comprised of a plurality of electrodes that are connected directly or indirectly to one another by conductive material. The electrode array includes zero, one or a plurality of centrally aligned electrodes 356, and zero one or a plurality of laterally arranged electrodes 352. One or more of the electrodes is directly or indirectly connected to the docking apparatus 360 via a ribbon interface 354 with embedded conductive material such that electrical signals may be passed on to the acquisition device once it is properly seated in the docking apparatus 360 via conductive elements 358. Each electrode includes a layer of medical-grade adhesive on the surface of the electrode that is in contact with the examinee's skin. In one embodiment of the disclosed system, the ribbon interface 354 that connects each electrode may also include a layer of medical-grade adhesive material.

In an exemplary embodiment of the disclosed system, the electrodes 352 are customized in form and number to allow for stable recordings across the frontal and anterior temporal regions in the FP1,2; AF7,8; F7,8 positions, defined by customary EEG lead placement, as understood by EEG practitioners. The sensors 352 do not contain latex and the adhesives used are all medical-grade, similar to adhesives commonly used in clinical situations such as achieving adhesion of electrocardiography electrodes or leads. In this same exemplary embodiment, each array of sensors is supplied in its own packaging and is disposable (e.g. not for reuse). The device, in some embodiments, is not implanted. Additional sensors may make contact with the skin contiguous with the EEG electrodes or via another surface of the body, for example with a second electrode or electrode array placed on the chest.

Each electrode 352 measures voltage differences between electrodes on the scalp (electroencephalogram). These measurements are in the millivolt (mV) range. In another embodiment of the disclosed system, the assembled head-mounted device also measures ambient light and noise levels.

In the embodiment depicted in FIG. 3B, the sensor is comprised of 8 EEG electrodes that are arranged in an array to cover the subject or patient's forehead. The electrodes are comprised of materials that are maximally breathable and lightweight to promote long-term wearability without undue discomfort to the examinee. In some embodiments, each electrode contains a non-latex adhesive layer that enables rapid application of the electrode to the examinee's forehead with little to no residue deposition when the electrode is removed. In some embodiments, each electrode also contains a thin ultra-lightweight electrically conductive component that is engineered to detect electrochemical depolarizations of the underlying central nervous system tissue, including the brain.

In other embodiments, the number or the arrangement of the electrodes may be different. For example, one embodiment may include only 3 electrodes, with 2 electrodes placed on the lateral aspects of the patient's forehead (left and right, respectively) and one electrode placed at the midline of the patient's forehead. In each of these embodiments, some subset of the electrodes may be used as ground or reference electrodes. In another embodiment, the device is provided to the operator, subject or other practitioner with multiple sensor devices of different sizes or electrode arrangements to accommodate a variety of clinical uses, or variation among the size or proportions of the examinee' forehead. In other embodiments, additional electrodes may be applied to other areas of the scalp or adjacent skin, such as behind the examinee's ear.

FIG. 3C is a detailed view of an exemplary construction of the printed electrode elements. In an exemplary embodiment of the device, the conductive material 362 that comprise the electrode detectors components are printed onto a flexible synthetic base 364. Additional circuit track components 366 are printed on said flexible synthetic base in order to carry electrical signals from the distributed electrodes to the docking apparatus and subsequently the transmitter device. In said embodiment, additional isolation layers are printed to separate the circuit tracks and prevent signal degradation or interaction between parallel tracks.

FIG. 3D is a detailed view of an exemplary assembly of the non-printed electrode elements. In said exemplary embodiment of the electrode array, the electrodes further comprise foam elements surrounding the electrode surface 370 and conductive foam elements that are centered on the electrode 372. In said embodiment, tines and medical-grade adhesive are used to secure low density gel sponges 368 into the center of each electrode. In said embodiment, electroconductive adhesive gel is used to enable the low-density sponge element to adhere to the subject's forehead. All components in said embodiment are medical-grade and tested to ensure absence of in vitro toxicity and absence of in vivo skin sensitization.

FIGS. 3E-F is an exploded (E) and an assembled view (F) of the docking apparatus that seats the acquisition device. The acquisition device docking apparatus functions to conduct electrical signals collected by the electrical array depicted in FIGS. 3B-D and to transmit said signals to the acquisition device for pre-processing and transmittal to the receiver device. The docking apparatus provides physical support for the acquisition device, including a rigid or semi-rigid supportive backing 382 that is contiguous with the base 384 of the docking apparatus. These components contain electrical components that enable signal conduction from the electrode array in contact with the examinee's skin to the electrically conductive components of the docking plug 388 that inserts into the acquisition device. In one exemplary embodiment of the docking apparatus, a grip component 386 clicks into the docking apparatus base 384 to provide the examinee or examiner an easily graspable component to steady the docking apparatus while applying the electrode array to the examinee's forehead or while the acquisition device is docked as shown previously in FIG. 3A with techniques 306 and 334 respectively.

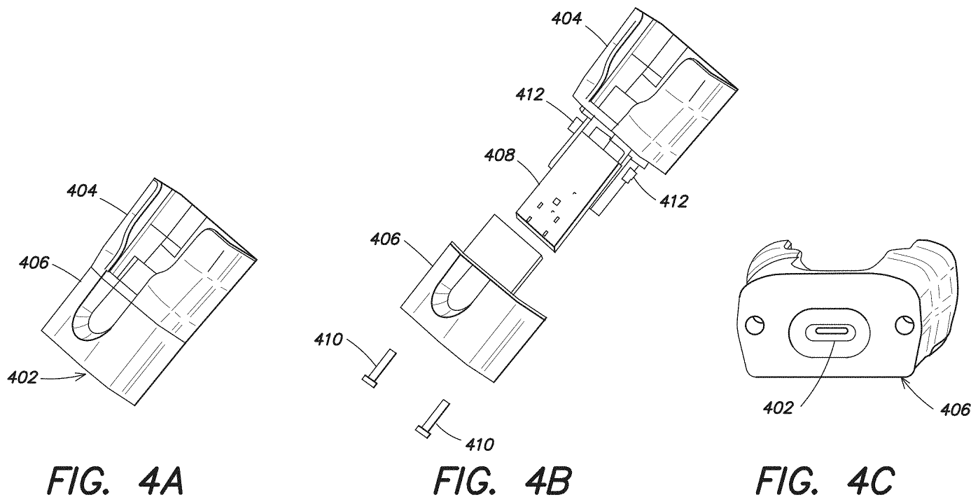

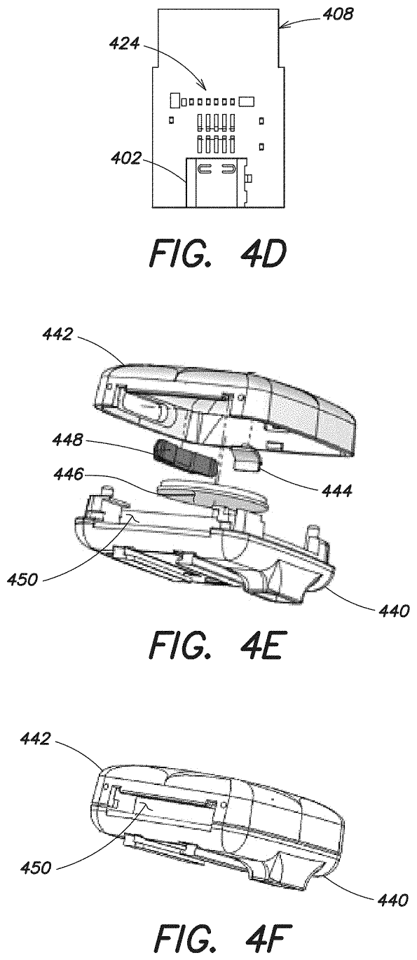

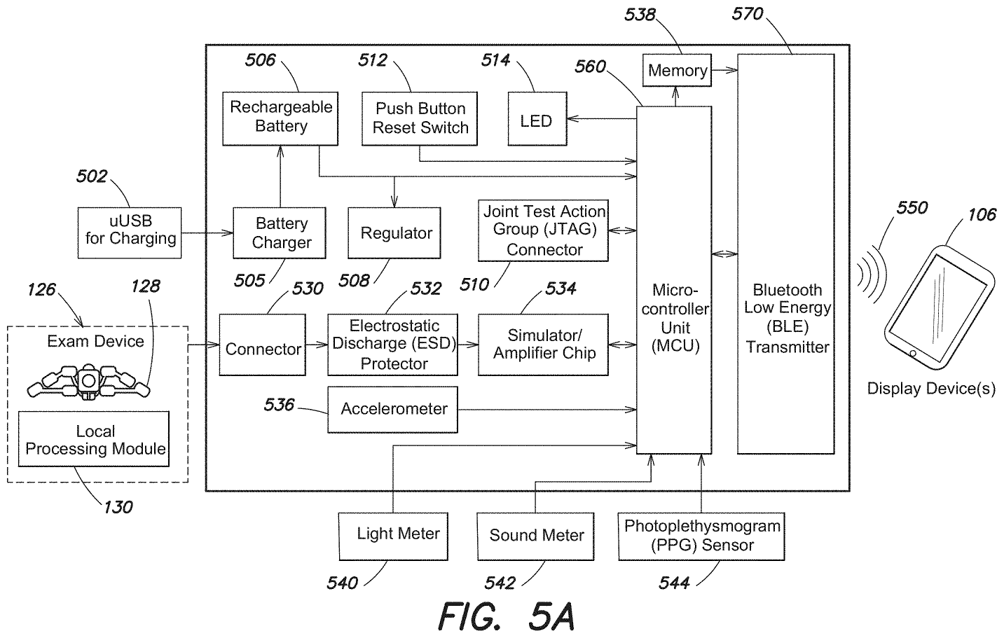

FIGS. 4A-C is a side-angle assembled view (A) and an exploded view (B) and an inferior view (C) of the charging apparatus. The charging apparatus is a separate component that enables electrical current to be conducted from a power source such as a wall outlet to the acquisition device in order to charge the integrated battery. The charging apparatus is primarily comprised of a power supply socket 402, external casing comprised of upper 404 and lower 406 components, and an integrated charging circuit board 408. The exploded view in FIG. 4B demonstrates how screws 410 are used to fasten (via receptacles 412) the upper 404 and lower 406 outer casing components in order to house the charging circuit board 408. The charging apparatus is intended to dock an acquisition device that does not have a fully charged battery component. The charging apparatus is not physically integrated with the EEG detector that is applied to the examinee's forehead.

FIG. 4D is a view of the charging apparatus circuit board. The circuit board 406 includes a power supply socket 402 that is compatible with a cord, such as a USB-C cord that is connected to one of a plurality of possible power sources, including but not limited to: a desktop computer, a laptop, a tablet, a phone or a wall outlet. The charging apparatus circuit board also contains electrically conductive components 424 that insert into the acquisition device to supply the electrical current from the power supply socket 402 and facilitate charging of the acquisition device battery. Alternatively, the docking apparatus may be used to upload firmware code to the acquisition device.

FIGS. 4E-F is an exploded view (E) and an assembled view (F) of the acquisition device. In an exemplary embodiment of the system, the acquisition device is completely sealed within casing components 440 and 442, with an integrated circuit board containing a digitization and wireless transmission module. In said embodiment, the recording device contains a 16-bit analog-to-digital converter with a voltage step size of 0.2 uV in order to pre-process analog signals gathered from the electrodes described in FIG. 3B into a format that is transmitted to the receiver device via a wireless format such as Bluetooth. In said embodiment, the acquisition device is powered by an integrated, rechargeable battery 446 that enables the apparatus to run continuously for over 24 hours while recording on all 8 channels. In said embodiment, the acquisition device is lightweight such that the entire EEG recording device including electrode array, docking apparatus and acquisition device weighs approximately 14 grams or less.

The acquisition device is enclosed by two casing components that form the anterior 442 and posterior 440 of the acquisition device, that are fastened by press-fitting the casing components together. In other embodiments, the casing may be fastened with screws, interlocking elements, adhesives or other elements to hold the casing together. In other embodiments, the casing and the internal components may be manufactured such that the casing is created in one continuous component, such as by 3D printing. In each of these embodiments, the casing is intended to be water resistant such that wiping down with standard cleaning wipes or other cleaning protocols may be performed without introducing moisture that clouds damage the internal electronic components.

In one exemplary embodiment, the anterior casing component includes a circular opening for a central semi-translucent diffuser component that is made accessible to the examinee or examiner on the front of the acquisition device. An additional optical conduction element 444 enables light generated by an LED indicator to be viewed from the anterior aspect of the acquisition device. In another exemplary embodiment of the disclosed system, multiple such LED indicators are included and produce light of one or more different colors, brightness, or duration. In another exemplary embodiment of the disclosed system, the central semi-translucent diffuser component may alternately serve as a button that the examiner or examinee can press to initiate a preset action. In yet another exemplary embodiment of the disclosed system, a button 448 is integrated into the lateral aspect of the anterior casing to enable one or more components that may be used by the examiner or examinee to control the function of the acquisition device. These one or more buttons may be used individually or in conjunction, for example by pressing together, to perform any one of a plurality of functions including but not limited to: powering the acquisition device on or off, initiating pairing of the device with the receiving device, pairing the device with the device electrodes or other sensors, initiating recording of signal from the electrodes or other sensors, identifying a significant clinical event such as a seizure or symptom experienced by the examinee, or cycling through any one of a plurality of detection modes or pre-defined programs or recording sequences.

In another embodiment, the central aspect or other aspect of the acquisition device includes an organic light-emitting diode (OLED) screen or other type of screen that is capable of displaying pre-programmed or generated visual graphics and is also capable of registering touch such that the examiner, examinee or other operator may provide input to control the system, for example to initiate an exam.

The acquisition device also contains a docking socket 450 which is used to dock the acquisition device onto the docking apparatus described in FIGS. 3C-D and demonstrated in FIG. 3A to facilitate use of the device for the performance of an examination. The docking socket 450 may alternatively be used to dock the acquisition device on the charging apparatus described in FIGS. 4A-D to facilitate charging of the battery integrated into the acquisition device, to download data or to upload firmware. In an exemplary embodiment of the disclosed system, the acquisition device automatically powers on when properly docked onto the docking apparatus as described in FIGS. 3C-D. Alternately or in addition, proper docking onto the docking apparatus may initiate pairing with the receiving device with or without additional input from the user via a user interface on the receiving or display device, or via pressing one or more of the buttons on the acquisition device. In an alternate embodiment of the disclosed system, the acquisition device may be paired by an action taken on the display device, including but not limited to tapping a user interface element within an app, taking a picture of a QR code or entering an alphanumeric code or pin that identifies the acquisition device and initiates a pairing sequence.

In an exemplary embodiment of the disclosed system, the acquisition device also includes the following components: a sound level sensor, a light level sensor, an oximeter, one or more light emitting diodes (LEDs), accelerometers, gyroscopes, and/or temperature sensors. In said exemplary embodiment, the sound level sensor is able to detect sound across a range of amplitudes with at least resolution (identified in decibels) and a sampling rate that may be adjusted to optimize for data quality, memory capacity, data relay and transmission bandwidth and/or the sampling rate of other integrated sensors. In said embodiment, the sampling rate may be modified to capture specific changes in sound levels, including but not limited to: snoring detection; respiratory rate detection; and ambient noise of the ICU/clinical setting. In said embodiment, the light level sensor contains a photodiode capable of sensing a dynamic range from light sources that may be adjusted and/or optimized depending on the exam environment. In said embodiment, the oximeter is capable of performing reflectance oximetry using integrated LEDs and a photodiode in order to compute oxygen saturation as a percent (SpO2) or other derivative values, such as total oxygen carrying capacity. In said embodiment, one or more indication LEDs are configured to show the status of the patient, status of the data collected by the electrodes and acquisition device and/or the status of the acquisition device. In said embodiment, different colors, brightness, blink frequency or patterns across multiple indicator LEDs may be implemented to communicate different signals to the examinee or examiner including but not limited to: signal quality, presence or absence of an EEG signature, pattern or diagnostic correlate such as delirium or epileptiform activity, battery charge level or connectivity to the receiver device. In said embodiment, an integrated accelerometer is able to detect head position and movements across 3 axes vertical (up and down), lateral (side to side) and horizontal (forward and backward). The gyroscope is configured to detect rotations, including pitch, yaw and roll as understood by standard notation in aviation.

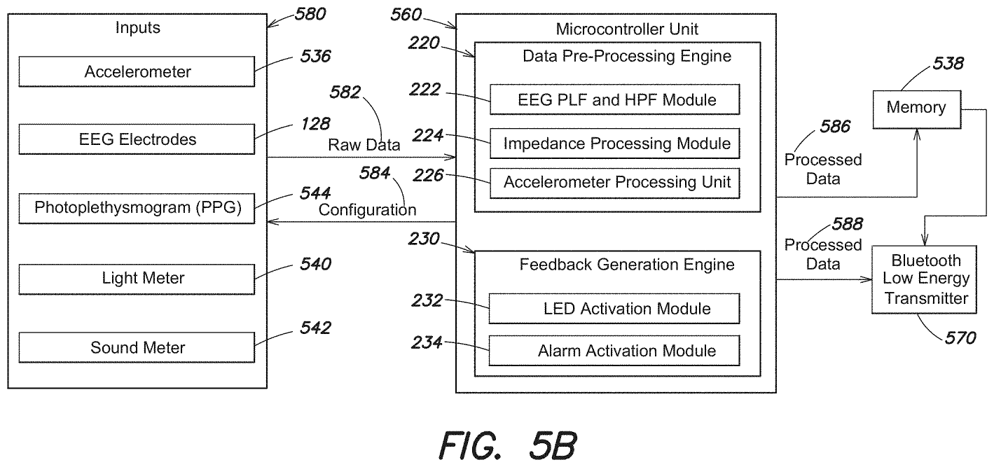

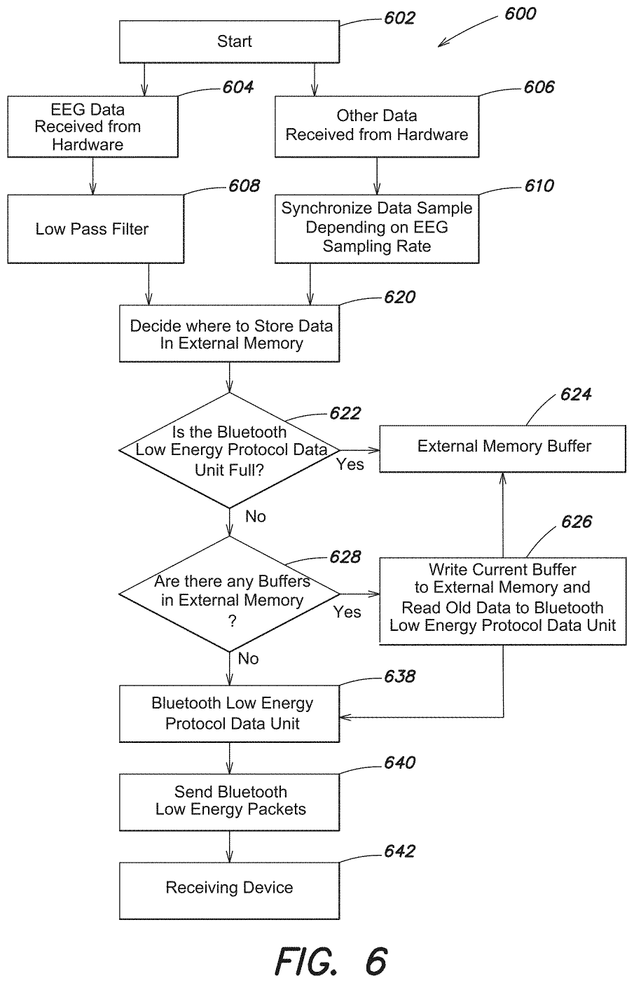

FIG. 5A is a diagram showing one embodiment of the local processing unit within the acquisition device. The local processing unit includes a rechargeable battery component 506 that carries enough charge to operate the acquisition device for at least 24 hours. The unit may also include a regulator 508 that regulates power to the device. In one exemplary embodiment of the device, the battery component is recharged (e.g., by microUSB, or other connection 502) by docking the acquisition device in the charging apparatus described in FIGS. 4A-D and connecting the docking apparatus to an appropriate power supply.

The Bluetooth low energy (BLE) transmitter 570 is capable of lossless wireless transmission 550 either directly to the display device 106 or to an alternate receiver device. In other embodiments of the disclosed system, this range may be extended by relay devices, by utilizing Wi-Fi or other wireless transmission protocol, or by using Bluetooth transmitter devices capable of longer-range transmission. In one exemplary embodiment, a time stamp is provided with each transmission of signal from the acquisition device to the receiver device. In said embodiment, the acquisition device is also capable of transmitting data relating to the function of the acquisition device itself, including but not limited to: remaining battery life, current sampling rate, signal capture efficiency, transmission fidelity, or other statistics related to the acquisition device's function. In said embodiment, changes in the EEG signal detected and processed by the acquisition device are converted into changes in the brightness, pattern or color of illumination of integrated LEDs (e.g., LED 514).

In an exemplary embodiment of the disclosed device, the acquisition device and the electrode array are capable of recording 0.3-100 Hz bandpass at a high fidelity, real-time 250 Hz sampling rate with a ground noise of 2.4 uVrms, an assumed electrode noise of 10 uVp-p, programmable hardware high-pass filter of 0.1 Hz-500 Hz, programmable hardware low-pass filter of 100 Hz-20 KHz, analog/digital resolution of 16 bits and an input dynamic range of ±6.5 mV.

In some embodiments, the acquisition device is capable of continuously transmitting pre-processed signal collected from the EEG leads. Alternatively, the acquisition device is capable of recording data in bulk and offloading defined segments of data, for example 30 minute or 24-hour intervals at specified timepoints during or after recording. In said embodiment, the data are encrypted and transmitted in an encrypted format such that any non-authorized device would be incapable of interpreting the transmitted data, including EEG signals, patient ID, device ID or personally identifiable health data, or any other data type communicated by the system.

The acquisition device includes various components, including, but not limited to a push button reset switch 512 for resetting the device, and one or more indicators, such as LED 514. The system may include one or more connections to external sensors or components, such as connection to the exam device 126 (and local processing module 130) through a connector 530, or other components through any number of connections, such as Joint Test Action Group (JTAG) connector 510. Further, the system may include any number of signal conditioning components, amplifiers or filters for conditioning any of the signals received. For example, signals fed through the connector 530 may be coupled to an ESD protector 532, and provided to a simulator/amplifier chip 534. Similarly, the system may have any number of on-board or external sensors such as accelerometer 536, light meter 540, sounds meter 542, photoplethysmogram (PPG) sensor 544. Input data received from these sensors may be fed to a Microcontroller unit 560 where the data is processed and communicated to the display device (e.g., display device(s) 106). Data, code, models and other data types may be stored in memory 538. In some embodiments, the local processing unit of the acquisition devices is fully programmable, and is capable of receiving and storing firmware updates.

FIG. 5B is a block diagram that depicts an embodiment of the software architecture that enables the acquisition device shown in FIG. 5A. In an exemplary embodiment, the primary inputs 580 for signal processing include data from the accelerometer 536, the EEG electrodes 128, the photoplethysmogram (PPG) 544, the light meter 540 and the sound meter 542. These input data are supplied to the microcontroller unit (MCU) 560 as raw or processed data streams 582. Simultaneously, the MCU 560 supplies configuration information 584 back to one or more of said sensors. The M726CU 560 is encoded with two primary engines, a data pre-processing engine 220 and a feedback generation engine 230. The data processing modules include EEG low-pass filters and high-pass filters 222, as well as processing modules for impedance 224 and accelerometer data 226. Said feedback generation engine 230 includes modules that control one or a plurality of LEDs 232 and a distinct module that controls one or a plurality of alarms 234. Processed data generated by said data pre-processing engine 220 a may either be communicated directly to the Bluetooth low energy (BLE) transmitter 570 via pathway 588 or alternately may be encoded to the local memory 538 via pathway 586 with or without subsequent communication to the BLE transmitter 570.

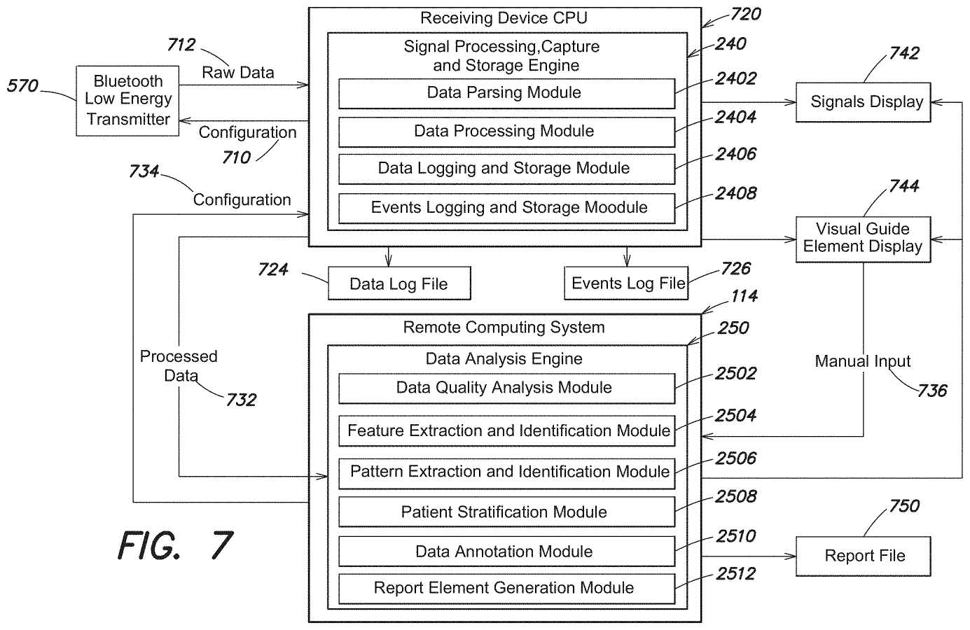

FIG. 6 is a flow diagram showing the acquisition and pre-processing of EEG data by the local processing unit within the acquisition device. Method 600 may be performed locally on an electronic device, online via a cloud system, or via some combination of the two. The method begins at 602. At 604, EEG data is received from the acquisition device. These data are then processed with the low pass filter at 608. In parallel or in sequence, at 606 other data are received from the acquisition device, including but not limited to light sensor data, sound sensor data, photoplethysmography data, accelerometer data or temperature probe data. At 620, the system decides where to store data in external memory. At 622, the system queries the blue tooth low energy (BLE) transmitter to determine if the protocol data unit is full. If the BLE protocol data unit is full then the data are sent to an external memory buffer 624. If the BLE protocol data unit is not full then at 628 the system queries whether there are any buffers in external memory. If there are buffers in external memory, then at 626 the system writes current buffers to external memory and reads old data to the BLE protocol data unit 638. If there are no buffers in external memory, then the system reads current data directly to the BLE protocol data unit 638. Subsequently at 640 the BLE protocol data unit sends BLE packets to the receiver device host 642. This method runs continuously while data are being collected by the acquisition device.

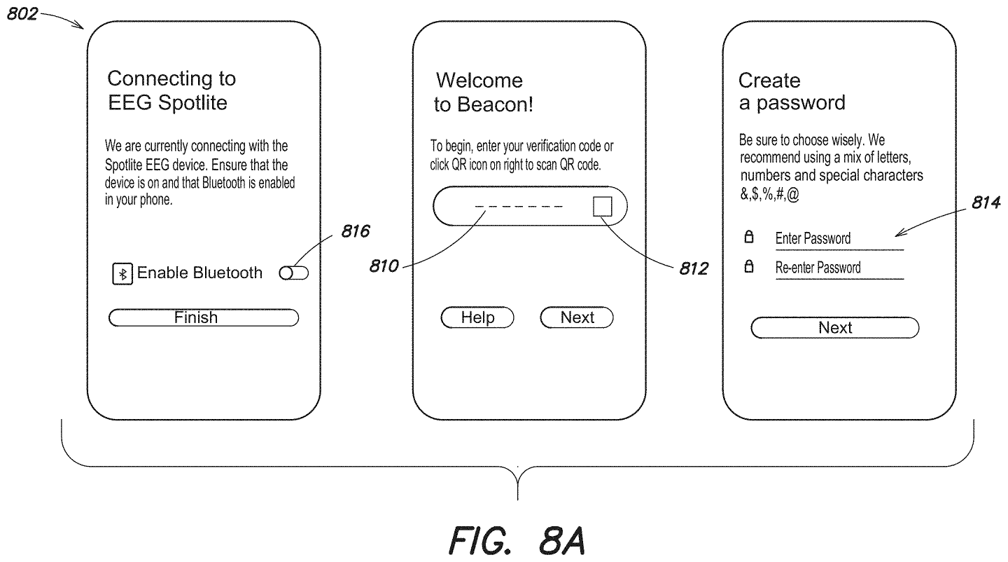

FIG. 7 is a block diagram that depicts an embodiment of the software architecture that enables the receiver device. In an exemplary embodiment, the receiver device and the display device are the same device. In other embodiments, the receiver device and the display device are separate devices.

In said exemplary embodiment where the receiver and display devices are the same device, the Bluetooth low energy transmitter 570 communicates data 712 to the receiving device processing unit 720 which implements the signal processing capture and storage engine 240. As previously described in FIGS. 2A-B, said signal processing capture and storage engine 240 enables implementation of a data parsing module 2402 and a data processing module 2404, which prepare data for further analysis or storage. The data logging and storage model 2406 and events logging and storage module 2408 interact with the storage layer 290 to capture and store data and events respectively, as determined by the requirements of the exam, or input from the examiner, examinee or other operator. Processed data are stored in a data log file 724 and events are stored in an events log file 726. Processed data may also be sent directly to a signal display interface 742 or to a visual guide element display 744. Alternately, processed data shown in path 732 may be sent to the data analysis engine 250. In one exemplary embodiment of the disclosed technology, the data analysis engine is implemented on a remote computing system 114. In another exemplary embodiment, the data analysis engine 250 is implemented on the receiver and display device. In yet another embodiment, some or all of the modules comprising said signal processing capture and storage engine 240 and said data analysis engine 250 are implemented on the local processing unit 130 of the head-mounted detector device. As described in FIGS. 2A-B, said data analysis engine 250 allows real-time or asynchronous assessment of output from the EEG detector device or other sensor (e.g., exam data), including data quality analysis 2502, feature extraction and identification 2504, pattern extraction and identification 2506, patient stratification 2508, data annotation 2510 and/or report element generation 2512. One or a plurality of analysis results generated by said data analysis modules working alone or in combination may be deposited in the form of a summary or detailed report in a report file 750 which is then stored in the storage layer 290. Generated report elements may be simultaneously or asynchronously created as a printed element, electronically generated in a static, dynamic, or interactive format, or may be modified into any other format by which the results of the exam may be communicated to the examiner, examinee, other operator, qualified expert, family member or other authorized party.

One or a plurality of analysis results generated by said data analysis modules working alone or in combination may alternately or simultaneously be communicated to the signals display 742 or to the visual guide element display 744. In an exemplary embodiment of the disclosed technology, the visual guide elements are displayed in conjunction with the underlying signal such that the visual guide elements highlight, annotate, or otherwise identify portions of the underlying data that contribute to the summary results of the analysis. Users may also provide manual input 736 in addition to any of the analysis (e.g., annotations or other type of input). Said visual guide elements may also indicate the confidence, strength of association or other indication of relativity with regards to the resulting assessment. One or a plurality of analysis results generated by said data analysis modules working alone or in combination may alternately or simultaneously be processed to supply configuration instructions 734 to said signal processing, capture and storage engine 240, or subsequently as configuration instructions 710 to the Bluetooth low energy transmitter 570 to update the instructions, function, methods, algorithms or other settings implemented by the local processing module 130 of the head-mounted detector device 126.

In the aforementioned exemplary embodiment where the receiver and display devices are the same device, the device is configured with sufficient local data storage for at least 14 days continuous recording within the storage layer 290. Alternately the receiving and display device is configured to offload portions of the data using communications network(s) 110 to a cloud storage solution to reduce the amount of data that is stored locally. In said embodiment where data is offloaded to a cloud storage solution, the duration of data that may be stored by the system is limited only by the availability of cloud storage. For said local data storage, said cloud data storage, or other form of data storage, clinical exam data are stored using a common standardized format and encryption protocol such that the same data ingestion and interpretation methods may be used to post-process data stored by multiple instances or embodiments of the disclosed system. The receiver and display device 106 as disclosed herein is also capable of initiating and maintaining real-time data streams of signals that are collected from the acquisition device which are then communicated to a cloud-based computing and storage platform, either directly over an encrypted connection 110 if external network access is available, or via protected intra-network endpoints that securely relay necessary outgoing/incoming connections. Said device is enabled with encryption algorithms to reconstitute data communicated by the acquisition device and/or to encrypt data that is subsequently communicated to other components of the system, such as a cloud-based remote computing module or data storage solution 118. Said encryption algorithms are of sufficient quality to prevent any unauthorized third party from intercepting and reconstituting transmitted data into a usable form that would in any way contain personally identifiable health data or any data on the status or function of any aforementioned components. In the circumstance that a third party attempted to intercept said encrypted data transmission the system is capable of recognizing such interference, halting data transmission and notifying the examinee, examiner, other operator or system administrator of said interference. Said device is enabled with Wi-Fi, a cellular SIM card, or any other communication device to communicate with said cloud storage solution and for other communication with central data repositories 118 or computing systems 114 as described in FIG. 1. In said embodiment, the receiver and display device 106 also contains a camera that may be used in addition to or instead of the integrated light meter on the acquisition device to detect ambient light or other light-based signals. Examples of hardware that would enable said exemplary embodiment includes but are not limited to a tablet computing device or a smartphone device.

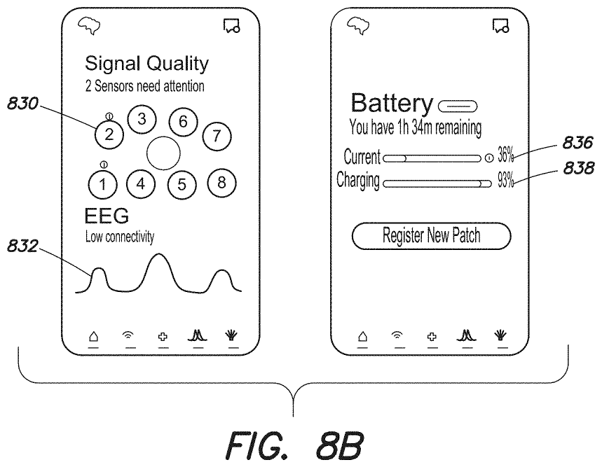

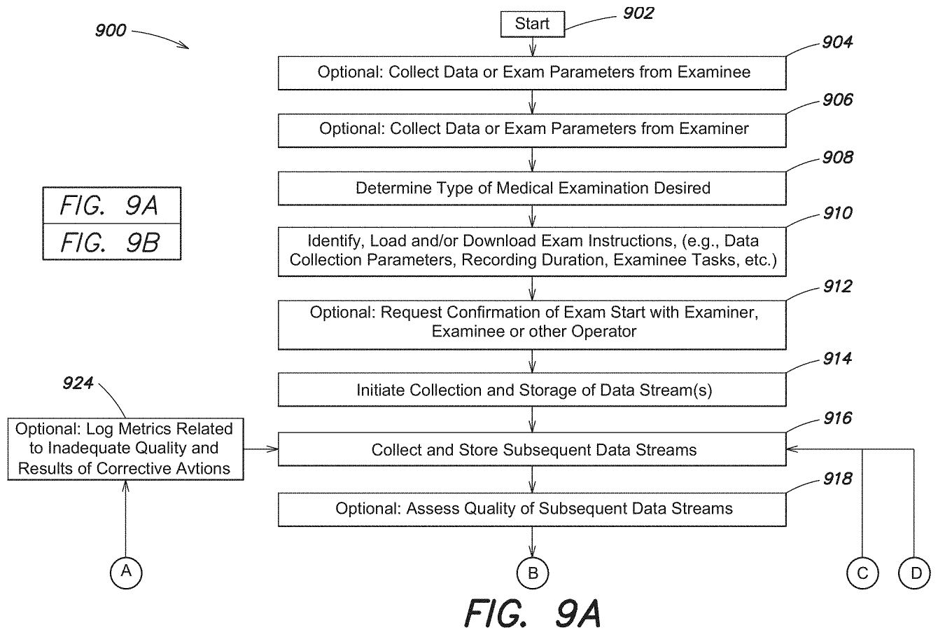

FIGS. 8A-B are diagrams demonstrating multiple methods of enabling pairing between the display and receiver device and the EEG acquisition device and feedback display elements that enable the examiner, examinee or other operator to interpret the current state of the acquisition device.

In said exemplary embodiment where the receiver and display devices are the same device, the receiver and display device is configured with a touch screen 802 to enable the examiner to interact with the system to input data including but not limited to the type of exam to be performed, adjunctive data about the patient or the clinical context, or to initiate the start of an exam. The receiver device and the acquisition device may be paired by one or more of a plurality of pairing procedures. One pairing procedure consists of the examiner, examinee or other operator initiating the pairing process using a search function on the display device, whereby the display device is first activated to access the appropriate communication method, for example Bluetooth 816, and subsequently provides a list of acquisition devices that are available for pairing and the examiner, examinee or other operator selects the device that he or she wishes to pair. Another pairing procedure is initiated by pointing the display device's integrated camera at a QR code captured with a camera initiated for example by tapping the graphical interface at 812, where the QR code is constructed to communicate the identity of the associated acquisition device, thereby enabling the display device to select the correct device from the list of possible in-range devices. In yet another pairing procedure, the examiner, examinee or operator is provided with an alphanumeric code entered for example at 810 that identifies the acquisition device from a list of in-range devices. In yet another pairing procedure, removal of a sticker, packaging or other disposable component from the electrode array or the acquisition device causes the acquisition device to become available for pairing with an in-range display device. One or more of these procedures may be used to exchange one acquisition device for a replacement acquisition device if the former acquisition device requires replacement, for example due to insufficient battery charge. The system enables the examiner, examinee, other operator or qualified expert to establish secure access via username and password combination 814, biometric data or 2-factor authentication to improve security of the examinee's personal and exam data.

In said exemplary embodiment where the receiver and display devices are the same device, the receiver and display device are capable of displaying EEG 832, accelerometer, light sensor, sound sensor or photoplethysmogram signals in real-time using a customizable layout montage. Said embodiment also includes real-time display of continuous impedance measurements. The examiner, examinee or other operator would have the ability to turn sensor channels on or off 830 depending on which data types are considered relevant. The interface would enable the examiner, examinee or other operator to control signal processing settings, including but not limited to values for gain, sampling rate and bandpass filters. The graphical user interface informs the examiner, examinee or other operator of the battery life remaining for the acquisition device 836 and for the receiver and display device, and also provides warnings and alarms if the acquisition device is falling out of range of the receiving device. In one embodiment, the graphical display would also indicate the battery charge status of an alternate acquisition device 838 that is paired, but not currently in use.

In some embodiments, the receiver and display device also includes interactive graphical display elements that enable the examiner, examinee, other operator or qualified expert to annotate time points, time periods, features, signatures or other elements of the incoming data stream that are relevant to the exam being performed with the system. Said interactive graphical display elements are customizable and are tailored to the specific exam being performed.

The receiving and display device is capable of providing configuration instructions to the acquisition device in the form of firmware updates that can subsequently be executed by the acquisition device microcontroller unit.

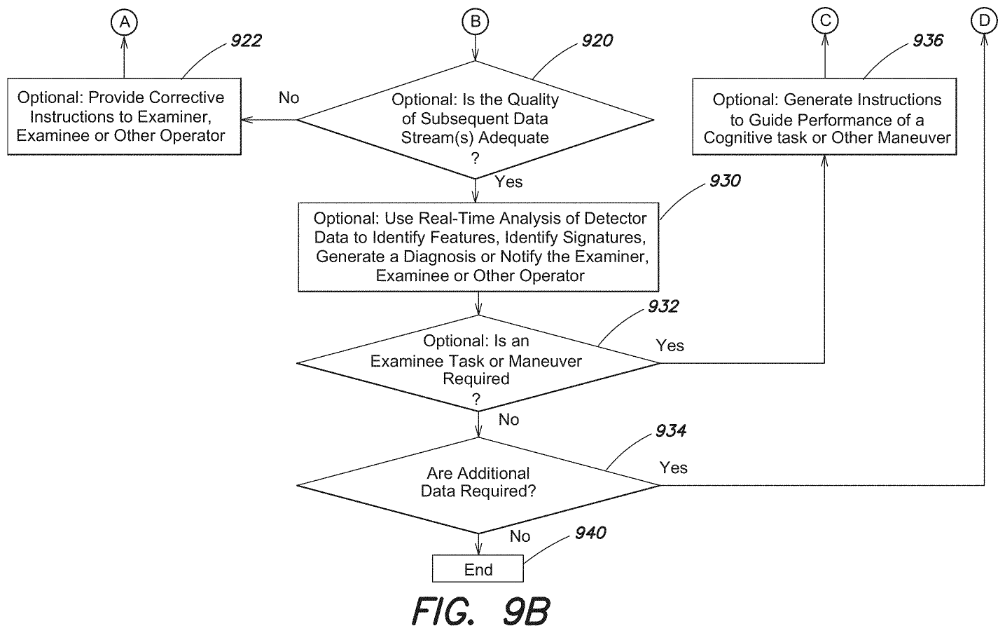

FIGS. 9A-B show a flow diagram demonstrating the initiation of an exam and collection of exam data enabled by the disclosed system and method. Method 900 may be performed locally on an electronic device, online via a cloud system, or via some combination of the two. The method begins at 902. At 904, the system optionally collects data or exam parameters from the examinee. An example of data collection in this step includes the examinee verifying their name or identity. At 906, the system optionally collects data or exam parameters from the examiner, or from another operator, technician, expert or other authorized individual. An example of data collected in this step includes the type or duration of exam to be performed. At 908, with or without input from the examinee, examiner, other operator, or other authorized individual, the system determines the type of examination to be performed. At 910, the system identifies, loads and/or downloads exam instructions, including but not limited to data collection parameters, recording duration, examinee tasks, hardware configuration or other such parameters. At 912, the system optionally requests input from the examiner, examinee or other operator to start the exam. At 914, the system initiates the collection, processing and storage of one or a plurality of data streams, and continues to 916 to collect, process and store subsequent data streams.

At 918, the system may optionally assess the quality of one or a plurality of data streams. Step 918 may be performed locally on an electronic device, online via a cloud system, or via a combination of the two. In some embodiments, the system assesses the current data streams for global quality metrics (e.g., signal to noise ratio, signal fidelity across electrodes, signal correlation across electrodes etc.). In said embodiment, the system extracts features and/or patterns that are relevant for quality assessment and compares the extracted features and/or patterns to data stored in a database or repository and/or that have been assessed by individuals skilled at EEG interpretation. In said embodiment, the system may integrate global data quality assessment metrics and feature and/or pattern comparisons against stored data to create an integrated score of the current image quality using a predefined threshold and/or threshold(s) derived from cumulative assessment across images in a database or repository. Alternately, the system may employ machine learning methods. For example, a machine-learning algorithm could be designed to function as an integrated classifier and grade images as “adequate” or “inadequate” based on features learned from a training database composed of images previously assessed by individuals skilled in EEG interpretation. Said methods could similarly be applied to data streams from one or a plurality of sensors, including but not limited to accelerometers, oximeters, temperature probes, gyroscopes or other sensors. In another embodiment, said methods are used to create mixed models that generate outputs based on a plurality of sensor inputs or clinical metadata such as lab values, demographics, radiology reports, pathology reports, clinical assessments or other clinical data. At 920, if the result of said analysis is that the quality of one or a plurality of data streams is determined to be inadequate, then at 922, the system may optionally provide corrective instructions to the examiner, examinee or other operator. Subsequently, at 924, the system may optionally log metrics related to the assessment of inadequate quality, the corrective action prescribed and the results of any corrective actions taken, prior to returning to step 916 to continue data acquisition. If the corrective action has not improved the quality of the exam data stream(s) then steps 918, 920, 922 and 924 may be repeated until at step 920 the data are determined to be of adequate quality.

If the system determines at step 920 that the quality of one or a plurality of subsequent data streams are adequate then the system may optionally use real-time analysis of detector data to identify features, identify signatures, generate a diagnosis, or notify the examiner, examinee or other operator of the results of the analysis (e.g., at step 930). In one embodiment, some portion or all of the signal interpretation analysis occurs on the local processing unit (e.g., using the microcontroller unit described in FIG. 5B). In said embodiment, the results of this analysis on the local processing unit are then sent to the receiver and display device. Alternately, the results of analyses performed by the local processing unit are used to activate or control the relevant feedback modules 230, in such a way that a feedback signal is generated including but not limited to a dynamic change in the LED to indicate detection of a feature or pattern. One example of a feature is an epileptiform spike. An example of a pattern is burst suppression. The local processing unit 130 may be programmed to generate specific feedback signals based on the type, frequency, duration or other parameter of the feature or pattern detected, for example by illuminating one or more LED's to indicate seizure activity, by generating a buzz to indicate a pending seizure, by illuminating an LED of a specific color or playing a specific sound to indicate that the examinee is a specified sleep stage such as rapid eye movement (REM) sleep. In another embodiment, a signal such as an illuminated LED, sound, or buzz is generated to indicate poor contact for one or more electrodes, or to indicate a corrective action required to improve signal quality.

In other embodiments, the results of said analysis are used to generate visual guide elements on the display device described in FIG. 1. In another embodiment, the results of said analysis may be utilized by the report element generation module 2512 described in FIGS. 2A-B to generate elements that may be included, record and logged in a summary or detailed report describing the results of the exam. In another embodiment, data pertaining to the quality of exam data or to the analysis of features, patterns, signatures or patient stratification interpretation may be communicated via a communication network to experts skilled in the art, such as neurologists, for manual interpretation or for verification of the accuracy of automated interpretation or annotation results. In said embodiment, the automated interpretation and annotation results could alternatively be used to calibrate or score the accuracy of expert interpreters, to train other individuals to perform interpretation or annotation at an expert level or to perform quality-control methods.

At 932, the system may optionally assess whether an examinee task or maneuver is required. Such an examinee task or maneuver may be pre-specified in the exam instructions or may be specified based on real-time analysis of one or a plurality of data streams. An example of an examinee task is to remember a number, word, or shape. An example of an examinee maneuver is to trace a figure. If such a task or maneuver is required, then at 936 the system may optionally generate instructions to guide the performance of a cognitive task or other maneuver and subsequently return to collecting data at 916. If multiple tasks or maneuvers are prescribed by the exam instructions, then the system subsequently returns to step 932 and determines that an additional task or maneuver is required. Said sequence may be performed one or a plurality of times. If no additional tasks or maneuvers are required, then the system assesses at 934 whether additional data are required. If required, the system returns to step 916 and continues to collect and store subsequent data. This sequence may be performed once or a plurality of times. If at step 934, no additional data are required then the method ends at step 940.

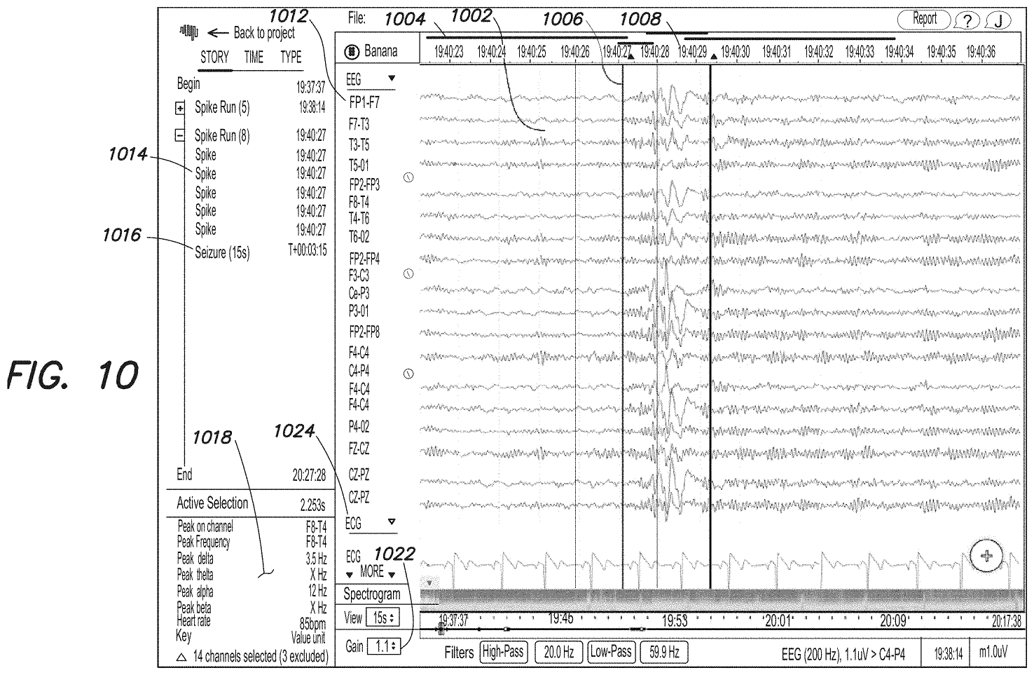

FIG. 10 is an exemplary embodiment of the display interface that would enable an expert to interrogate the data captured by the detection device with the assistance of virtual guide elements and the ability to annotate or confirm diagnostic interpretations.

In said exemplary embodiment, an expert is presented with a virtual dashboard that enables visualization and navigation of the raw recorded data 1002, optionally segmented or organized by the relevant electrode 1012 or alternate data stream, such as an electrocardiogram lead 1024. The interface displays visual guide elements, such as automated annotations 1004 that are created by statistical models to identify segments of raw data that have a given probability of identifying diagnostically relevant information. Other virtual tools 1006 enable the operator to manually annotate segments of interest to add expert interpretation or to confirm automatically annotated interpretations. The data is viewed with relevant time stamps 1008 enabling ease of navigation. In said exemplary embodiment, the interface also displays a consolidated log of events or spans of diagnostic relevance 1014, which may include automated annotations, manual annotations or both. Said element in the interface may also display a diagnostic interpretation such as “seizure” 1016, which may similarly be generated by automated interpretation, manual interpretation or a combination of automated and manual inputs. An additional interface element 1018 summarizes overall statistics from the relevant collected data or any defined subset thereof. In said embodiment, the interface also includes additional tools to define key signal processing parameters 1022 including but not limited to: gain, high-pass filter value, low-pass filter value or time window to analyze.

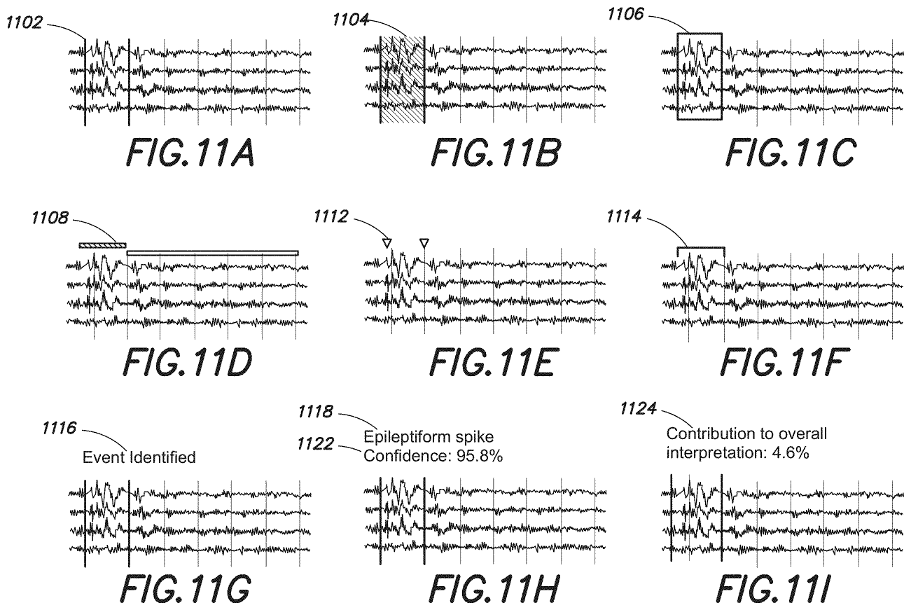

FIGS. 11A-I are examples of virtual guide elements that are used to identify features or signatures for the purpose of identifying diagnostically relevant signals or patterns.

The system and method described herein is capable of generating a wide range of virtual guide elements. Said elements may be generated as the output of a statistical model trained on a large volume of EEG data in order to identify segments of data of likely diagnostic importance, or to directly annotate said segment with a diagnostic interpretation. Alternatively, an expert human interpreter may generate a virtual guide element to mark his or her own expert interpretation or annotation such that another operator, another expert, the patient or the original expert may subsequently view the virtual guide element. Virtual guide elements may appear in a wide range of form-factors, including but not limited to the exemplary embodiments illustrated in FIGS. 11A-I. In FIG. 11A, the virtual guide element 1102 employs boundaries to highlight the data span of interest. In FIG. 11B, the virtual guide element 1104 employs a transparent colored or textured element to highlight the data span of interest. In FIG. 11C, the virtual guide element 1106 employs an enclosed object to highlight the data span of interest. In FIG. 11D, the virtual guide element 1108 employs a superimposed band to highlight the data span of interest. In FIG. 11D, the virtual guide element 1108 employs a superimposed band to highlight the data span of interest. In FIG. 11E, the virtual guide element 1112 employs superimposed pointers to highlight the data span of interest. In FIG. 11F, the virtual guide element 1114 employs a superimposed bracket to highlight the data span of interest. In FIG. 11G, the virtual guide element 1116 employs text in combination with geometric virtual guide elements to annotate the data span of interest. In

FIG. 11H, the virtual guide element 1118 employs text to annotate the type of event identified, along with a text-based indicator of confidence 1122 in combination with geometric virtual guide elements to annotate the data span of interest. In FIG. 11I, the virtual guide element 1124 employs text to identify the relevance of the highlighted span for the overall interpretation of the data collected, along with an indicator of the relevant weight of the contribution.

In all of said exemplary embodiments of virtual guide elements, said elements may be colored, textured, sized, or otherwise highlighted or enhanced to make them visually appealing, easy to locate, or to communicate information such as event category. In another exemplary embodiment of the technology, said virtual guide elements are not static, but dynamically respond to the collection of data, addition of new annotations or other user or automated inputs.

It should be appreciated that any of the above-described embodiments may be practiced alone or in combination with any other embodiment(s). Further, it should be appreciated that any of the embodiments described herein may be practiced with any of the aspects described in the provisional patent application U.S. Ser. No. 63/075,711 filed Sep. 8, 2020 entitled “SYSTEMS AND METHODS FOR MEASURING NEUROTOXICITY IN A SUBJECT” which forms an integral part of the application and is hereby incorporated by reference by its entirety.

### EXAMPLE SYSTEM—MEASURING NEUROTOXICITY IN A SUBJECT

The following describes an example process using various embodiments of the computer-based system for determining neurotoxicity in a patient. History of seizures or epileptiform findings on EEG may be used as an exclusion criterion for clinical trials. Pre-enrollment EEG screens may be used for phase 1 trials in a wide range of neurological disease. In this setting, presence of epileptiform spikes are used to exclude patients from the trial or to establish precedent if a patient subsequently develops seizures following administration of a novel agent. Single neurologists serving as central readers currently use clinical judgement whether to put a trial on hold once abnormal activity is identified and alternate subjects are enrolled subject is enrolled. However, it is appreciated that there exists tremendous inter-rater disagreement on the identity of epileptiform activity patterns across skilled epileptologists. As a result, there has not previously been a way to objectively and rigorously follow trial exclusion protocols that could save lives, optimize management of neurotoxicity, and allow effective drugs to reach the market.

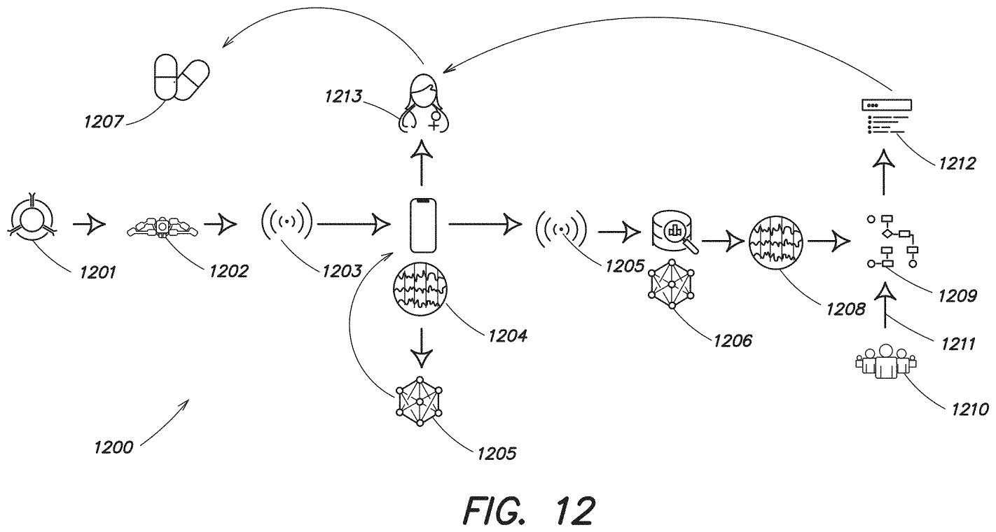

FIG. 12 shows one example process 1200 for detecting neurotoxicity in a patient/subject of a clinical trial using EEG data. At block 1201, process 1200 begins where a patient or subject in the clinical trial receives an experimental or commercial therapy (e.g. the FDA approved chimeric antigen receptor T cell (CAR-T) therapy for B-cell acute lymphoblastic leukemia treatment described below). At block 1202, an EEG device (e.g., a low-profile EEG device (recording EEG, sp02, accelerometer, gyroscope, light-meter, sound-level meter, thermistor, or plurality of sensors measuring other types of data)) is placed either after treatment initiation. Also, it should be appreciated that the data may be collected before the application of the therapy in order to get brain baseline activity and estimate toxicity risk score.

At block 1203, data acquired from the EEG device (e.g., including EEG or other types of data) are communicated to a computer system (e.g., a smartphone, computer, tablet, etc.) via a communication medium (e.g., wireless (e.g., Bluetooth, Wi-Fi, etc.) and/or wired (e.g., USB, Ethernet, etc.) connections). At block 1204, raw EEG data may be displayed on the computer system (e.g., a tablet) which can be used by clinician to make clinical decisions

At block 1205, the system may use machine learning or other AI mechanism to automatically annotate the EEG data. This can be accomplished on-device and/or by other systems (e.g., a cloud-based system or components). In some examples, machine learning algorithms may be used, such as CNNs, LSTMs, Transformer networks, etc., to annotate the EEG for high risk features of neurotoxicity and aggregate neurotoxicity risk scores that can be used by clinician/nurse (e.g., at block 1213) to guide an antidote or treatment to the toxic therapy or to mitigate the symptoms of novel therapy (e.g., at block 1207). Further, the system may send instructive messages such as an alert on the recording device or messages may be sent to the clinician directly via an EMR or smartphone.

The system may have one or more additional features, either alone or in combination with any feature described here. For instance, the system may alert the clinician/nurse or other user, such as by providing an alert on the recording device or sending an alert to the clinician directly via an electronic medical record (EMR), smartphone, or other method, or some condition, event, annotation or other occurrence. The device obtaining the data may communicate with an external device and/or cloud platform (e.g., using Wi-Fi) in real-time or alternatively there may be some delay in sending/receiving data. For instance, at some time later, the data may be sent to one or more systems (e.g., at block 1205).

In some implementations (e.g., at block 1206) machine learning analytics (single or multiple algorithms) operate on streaming or stored data to identify pathological features (e.g., epileptiform spikes, seizures, abnormal slow activity, machine learning derived features, etc.). Further, in some implementations, annotations of suspected pathological events may be stored, as are quantitative and qualitative toxicity risk scores are saved.

Further, a web-based portal may be provided (e.g., at block 1208). In some embodiments, the portal allows clinicians to login to view and interact with the data and analyses (e.g., at block 1210), and the clinician may be alerted that there is a new recording ready to review, and the system may track the clinician's review of the data. Further, a neurologist may be provided an opportunity to review raw data and accept/reject machine-learning annotations corresponding to possible neurotoxicity (e.g., through a portal interface at block 1211).

In some implementations, the system may provide automated or templated reports. For instance, a templated report may be generated from algorithmic or augmented labelling workflows (e.g., at block 1209) by automatically capturing features and images from the recordings into the PDF reports). Such reports may allow a clinician or neurologist more easily interpret significant data. Such reports may be managed by the system, finalized into a final form, and tracked by the system (e.g., at block 1212). Clinicians (e.g., at block 1213) may review such reports through a login to the system to perform review of the reports. Through the interface and by reviewing the report, a clinician is enabled to make informed clinical decisions. For instance, a clinician may decide to reverse neurotoxicity by removing the therapy (at block 1207), to guide an antidote or treatment to the toxic therapy, or to mitigate the symptoms of a novel therapy.

As discussed, one example where such systems may be useful includes measuring neurotoxicity of CAR-T therapy patients. In 2017, the FDA approved chimeric antigen receptor T cell (CAR-T) therapy for B-cell acute lymphoblastic leukemia (ALL) and in 2018 CAR-T therapy was approved for Diffuse large B-cell lymphoma (DLBCL). Rapidly, CAR-T therapy has also been shown effective in chronic lymphocytic leukemia (CLL) and non-Hodgkin lymphoma (NHL) and most recently mantle-cell lymphoma. However, CAR-T therapy can cause substantial morbidity and, rarely, mortality, due to cytokine release syndrome (CRS) and neurotoxicity, also known as CAR-T cell-related encephalopathy syndrome (CRES) or immune effector cell associated neurotoxicity syndrome. In a study of patients receiving CD19 CAR-T for B-ALL, CLL, and NHL, 40% of patients experienced grade 1 or higher CRES, and 3% died due to CRES, most often related to cerebral edema. ICAN can manifest as headaches, diminished attention, aphasia, seizures, status epilepticus and somnolence requiring intubation (Karschnia et al., 2019). Early detection is key, because CRES may respond to dexamethasone and possibly cytokine (IL6, IL1, JAK) targeted therapies such as siltuximab, a direct IL-6 blocking agent.

Accurate diagnosis and assessment of the degree of ICANS is also critical because prolonged steroid use may diminish the efficacy of CAR-T therapy against the underlying malignancy and potentially exacerbate delirium. Moreover, some research indicates that the preferred treatment for CRS, tocilizumab, may worsen CRES. There is, therefore, a critical unmet need for clinical grade technologies that can track and predict neurotoxicity. Although such systems may be used to measure neurotoxicity in CAR-T therapy situations, it should be appreciated that various embodiments may be used with other therapies and may be used to detect other neurological events.

### Additional System Features

It should be appreciated that certain system elements may be used alone and/or in combination with other elements, and that more or less steps may be performed in the process. Further, some embodiments relate to a novel patch design and associated elements for measuring EEG signals. However, it should be appreciated that other methods and devices for measuring EEG signals can be used.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware or with one or more processors programmed using microcode or software to perform the functions recited above.

In some embodiments, a machine learning model, artificial intelligence model or other type of statistical model may be used to determine environmental parameters to be used to process one or more EEG or other signal types. In one implementation, a model may be trained based on EEG signals. Certain outcomes, diagnoses, or other outputs may be also used to train the model and permit the model to predict outcomes based on the input parameters. The model may be part of a computer system used to provide indications to one or more users. Other implementations and systems can be used that take EEG signals to appropriately predict one or more outcomes. Analysis may be performed with or without the use of electronic medical record data, ingestion of other data streams (e.g., heart rate, temperature, and/or pulse oximetry) and/or a corpus of previously collected EEG data. A display device enables the operator to provide input to set the parameters of the exam. The same or a different display device provides real-time data feeds or summary analysis to an expert for interpretation and/or a healthcare provider responsible for care of the subject and/or the subject themselves for interpretation.

In this respect, it should be appreciated that one implementation of the embodiments comprises at least one non-transitory computer-readable storage medium (e.g., a computer memory, a portable memory, a compact disk, etc.) encoded with a computer program (i.e., a plurality of instructions), which, when executed on a processor, performs the above-discussed functions of the embodiments. The computer-readable storage medium can be transportable such that the program stored thereon can be loaded onto any computer resource to implement the aspects discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects.

Various aspects may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments may be implemented as one or more methods, of which an example has been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

The terminology utilized herein is intended to describe specific embodiments only and is in no way intended to be limiting of the invention. The term “and/or”, as used herein, includes any and all combinations of one or more of the associated listed items. As used herein, the singular forms “a,” “an,” and “the” are intended to include the plural forms as well as the singular forms unless the context clearly indicates otherwise. As used herein, the terms “comprises” and/or “comprising” are intended to specify the presence of features, elements, or steps, but do not preclude the presence or addition of one or more additional features, elements, and/or steps thereof. The term “device” as used herein should be interpreted as a broad term defined as a thing adapted for a particular purpose. This includes but is not limited to medical examination tools such as EEG electrodes, EKG electrodes, oximetry probes, temperature probes, but also other things a user may utilize in performing an exam such as a user's own hands, ears, nose, or eyes. The terms “electroencephalography electrode” or “EEG electrode ” should be understood to mean an electrode capable of detecting electrical activity, including depolarization and repolarization of neuronal tissue spatially arranged in the central nervous system, including various regions of the brain. Such electrical activity detected by an EEG electrode is generally thought by those skilled in the art to represent the summed electrical activity of a plurality of neurons acting synchronously or asynchronously individually, in groups, or in networks that are spatially arranged in the area detected by the electrode.

Unless defined otherwise, all terms used herein have the same meaning as commonly understood by one having ordinary skill in the art to which the invention relates. Furthermore, it will be understood that terms should be interpreted as having a meaning that is consistent with their meaning in the context of the relevant art and the present disclosure and will not be interpreted in an overly formal or idealized sense unless expressly defined herein.

To avoid confusion, this description will refrain from repeating every possible combination of features, elements, and/or steps. However, it will be understood that any and all of the listed features, elements and/or steps each has individual benefit and can also be used in conjunction with one or more, or in some cases all, of the other disclosed items, particularly in the claims. Thus, the specification and claims should be read with the understanding that any of these combinations are entirely within the scope of the invention and the claims.

What is claimed is:

1. An electroencephalography (EEG) detection system comprising:
a wearable head-mounted device, comprising:
a plurality of sensors arranged at different locations, with each sensor configured to capture electrical signals from a portion of a body of an examinee; and
a data acquisition apparatus configured to process electrical signals from the sensors and wirelessly transmit said electrical signals to a receiver device, the receiver device being configured with one or more processors to receive and process data transmitted by the acquisition device; and
one or more computer storage media to store data generated by the head-mounted device.

2. The EEG detection system of claim 1, further comprising a display device configured to present virtual content to the examinee, examiner or other operator.

3. The EEG detection system of claim 1, wherein said data acquisition apparatus continuously transmits said EEG signals to said receiver device.

4. The EEG detection system of claim 1, wherein said data acquisition apparatus is capable of being configured wirelessly.

5. The EEG detection system of claim 1, wherein a flexible material is used to connect the sensors in a pre-specified arrangement such that the examinee, examiner or other operator may rapidly apply the sensors at the desired locations without having to place each sensor individually.

6. The EEG detection system of claim 5, wherein said flexible material and said sensors may be manufactured in a plurality of arrangements, including variation in number and/or position of sensors, that may be interchangeably attached to the data acquisition device according to examinee characteristics, desired exam or clinical use case.

7. The EEG detection system of claim 1, wherein said head-mounted electrode array and said acquisition device are integrated into the same device.

8. The EEG detection system of claim 2, where said display device and said receiver device configured with one or more processors are the same device.

9. The EEG detection system of claim 1, wherein the head-mounted device is further configured with one or more sensors from the group comprising:
an oximeter;
a temperature sensor;
a gyroscope;
an accelerometer; and
a heart rate monitor.

10. The EEG detection system of claim 1, wherein said one or more computer storage media are further configured to store one or more from the group comprising:
a database for quality comparison;
a database for feature identification;
a database for pattern identification; and
a database for patient stratification and population analysis.

11. The EEG detection system of claim 10, wherein said one or more processors are further configured to perform operations comprising:
accessing one or more from the group comprising:
a database for quality comparison;
a database for feature identification;
a database for pattern identification; and
a database for patient stratification and population analysis;
assessing the quality of exam data;
extracting or identifying features from the exam data;
extracting or identifying patterns from the exam data; and
extracting or identifying patient stratification or other population analysis.

12. The EEG detection system of claim 11, wherein, responsive to the collection of additional data from the one or more examination devices or sensors, the one or more processors are further configured to perform operations comprising of one or more from the group comprising:
creating a report summarizing the results of exam data analysis;
creating data visual guide elements that are displayed to the user;
creating data annotations that are stored along with the underlying data in a non-transitory computer-readable medium; and
configuring the head-mounted device to provide feedback to one or more users in the form of light, sound, or vibration.

13. The EEG detection system of claim 12, wherein:
the one or more processors are further configured to perform train a machine learning or statistical model on data stored in a non-transitory computer-readable medium; and
the one or more computer media are further configured to store the model architecture, parameter values and any other variables required to implement said machine learning or statistical model.

14. The EEG detection system of claim 13, wherein, responsive to the collection of additional data from the one or more examination devices or sensors, the one or more processors are further configured to re-train said machine learning or statistical model with an updated data set.

15. A non-transitory computer-readable medium storing one or more instructions that, when executed by one or more processors, cause the one or more processors to perform operations comprising:
accessing one or more from the group comprising:
a database for quality comparison;
a database for feature identification;
a database for pattern identification; and
a database for patient stratification and population analysis;
assessing the quality of exam data;
extracting or identifying features from the exam data;
extracting or identifying patterns from the exam data; and
extracting or identifying patient stratification or other population analysis.

16. The non-transitory computer-readable medium of claim 15, wherein, responsive to the collection of additional data from the one or more examination devices or sensors, said non-transitory computer-readable medium stores further instructions that, when executed by one or more processors, cause said one or more processors to perform operations comprising of one or more from the group comprising:
creating a report summarizing the results of exam data analysis;
creating data visual guide elements that are displayed to the user;
creating data annotations that are stored along with the underlying data in a non-transitory computer-readable medium; and
configuring the head-mounted device to provide feedback to one or more users in the form of light, sound, or vibration.

17. The non-transitory computer-readable medium of claim 16, further storing instructions that, when executed by one or more processors, cause said one or more processors to:
train a machine learning or statistical model on data stored in a non-transitory computer-readable medium; and
store the model architecture, parameter values and any other variables required to implement said machine learning or statistical model.

18. The non-transitory computer-readable medium of claim 17, wherein, responsive to the collection of additional data from the one or more sensors, said non-transitory computer-readable medium further stores instructions that, when executed by one or more processors, cause said one or more processors to re-train said machine learning or statistical model with an updated data set.

19. The non-transitory computer-readable medium of claim 18, further comprising:
a remote data repository comprising the one or more computer storage media storing the computer readable instructions; and
a remote processing module comprising the one or more processors, wherein the one or more processors are configured to perform the operations.

20. A method, comprising:
determining the type of medical examination desired;
initiating collection and storage of data streams from said system;
collecting and/or storing subsequent data streams from said system;
accessing one or more from the group comprising:
a database for quality comparison;
a database for feature identification;
a database for pattern identification; and
a database for patient stratification and population analysis;
assessing the quality of exam data;
extracting or identifying features from the exam data;
extracting or identifying patterns from the exam data;
creating a report summarizing the results of exam data analysis;
creating data visual guide elements that are displayed to the user;
creating data annotations that are stored along with the underlying data in a non-transitory computer-readable medium; and
configuring the head-mounted device to provide feedback to one or more users in the form of light, sound, or vibration.

21. An electroencephalography (EEG) detection system comprising:
a wearable head-mounted device, comprising:
a processor;
a memory coupled to the processor; and
an array of sensors, operatively coupled to the processor and configured to capture electrical signals from a forehead area of a subject, wherein the array of sensors are located at fixed positions and are capable of being attached to the forehead area.

22. The system according to claim 21, wherein array of sensors are positioned within a flexible component that are capable of being attached to the forehead area by an adhesive layer.

23. The system according to claim 22, wherein at least of the sensors in the array of sensors includes an electrically conductive component configured to detect electrochemical depolarizations of underlying central nervous system tissue of the subject.

24. The system according to claim 22, wherein the array of sensors includes at least a first electrode positioned within the flexible component in a location that aligns substantially with a midline of the subject's forehead area.

25. The system according to claim 24, wherein the array of sensors includes at least a second and third electrode, each of which is positioned within the flexible component at lateral left and right locations relative to the midline of the subject's forehead area.

26. The system according to claim 21, wherein the array of sensors is a disposable item.

27. The system according to claim 21, wherein the array of sensors is detachable from the processor and memory.

28. The system according to claim 21, wherein the wearable head-mounted device further comprises a visual indicator that is configured to provide a visual indication to a person monitoring the subject.

29. The system according to claim 21, wherein the wearable head-mounted device further comprises an audio sensor that is configured to detect ambient sound in an environment of the subject.

30. The system according to claim 21, wherein the wearable head-mounted device further comprises a light sensor that is configured to detect ambient light in an environment of the subject.

31. The system according to claim 21, wherein the array of sensors includes a particular arrangement of sensors for a corresponding particular clinical use.

32. The system according to claim 21, wherein the array of sensors is configured to be applied to desired locations on the subject's forehead without having to place each sensor individually.

33. The system according to claim 21, wherein the wearable head-mounted device is configured to provide feedback to the subject in the form of light, sound, and/or vibration.

34. The system according to claim 21, wherein the electrical signals are EEG signals.

35. The system according to claim 21, wherein the head-mounted device is further configured with one or more sensors from the group comprising:
an oximeter;
a temperature sensor;
a gyroscope;
an accelerometer; and
a heart rate monitor.

36. An electroencephalography (EEG) detection system comprising:
a wearable head-mounted device, comprising:
a plurality of sensors arranged at different locations on a subject, with each sensor configured to capture EEG signals from the subject; and
a data acquisition device configured to process electrical signals from the sensors and transmit said EEG signals to a receiver configured with one or more processors to receive and process data transmitted by the acquisition device; and
a machine learning engine configured to receive and process the EEG signals and perform
at least one of the group of operations comprising:
automatically identify patterns within the received EEG signals;
automatically annotate at least a portion of an EEG waveform;
control a visual indicator to signal an examiner of the subject of a particular condition of the subject;
indicate a quality of the EEG signals; and
control a feedback generator to provide feedback to the subject in the form of at least one of the group comprising light, sound, and vibration.

37. The system according to claim 36, wherein the machine learning engine is configured to receive and process signals from one or more of the sensors from a group comprising:
an oximeter;
a temperature sensor;
a gyroscope;
an accelerometer; and
a heart rate monitor.
